SEQUENCE LISTING (270 sequences)

SEQ ID NO: 1 - protein, 11 aa, Bacillus thuringiensis
GSAQGIEGSIR

SEQ ID NO: 2 - protein, 11 aa, Bacillus thuringiensis
IVAQLGQGVYR

SEQ ID NO: 3 - protein, 8 aa, Bacillus thuringiensis
TLSSTLYR

SEQ ID NO: 4 - protein, 8 aa, Bacillus thuringiensis
DVSVFGQR

SEQ ID NO: 5 - protein, 5 aa, Bacillus thuringiensis
TYPIR

SEQ ID NO: 6 - protein, 7 aa, Bacillus thuringiensis
TVSQLTR

SEQ ID NO: 7 - protein, 8 aa, Bacillus thuringiensis
WYNTGLER

SEQ ID NO: 8 - protein, 12 aa, Bacillus thuringiensis
EWEADPTNPALR

SEQ ID NO: 9 - protein, 7 aa, Bacillus thuringiensis
VWGPDSR

SEQ ID NO: 10 - protein, 9 aa, Bacillus thuringiensis
APMFSWIHR

SEQ ID NO: 11 - protein, 11 aa, Bacillus thuringiensis
WGFDAATINSR

SEQ ID NO: 12 - protein, 6 aa, Bacillus thuringiensis
NQAISR

SEQ ID NO: 13 - protein, 6 aa, Bacillus thuringiensis
IEEFAR

SEQ ID NO: 14 - protein, 12 aa, Bacillus thuringiensis
SGFSNSSVSIIR

SEQ ID NO: 15 - protein, 8 aa, Bacillus thuringiensis
LSHVSMFR

SEQ ID NO: 16 - protein, 16 aa, Bacillus thuringiensis
EIYTNPVLENFDGSFR

SEQ ID NO: 17 - protein, 16 aa, Bacillus thuringiensis
LEGLSNLYQIYAESFR

SEQ ID NO: 18 - protein, 5 aa, Bacillus thuringiensis
YNQFR

SEQ ID NO: 19 - protein, 6 aa, Bacillus thuringiensis
YNDLTR

SEQ ID NO: 20 - protein, 19 aa, Bacillus thuringiensis
SPHLMDILNSITIYTDAHR

SEQ ID NO: 21 - protein, 20 aa, Bacillus thuringiensis
SAEFNNIIPSSQITQIPLTK

SEQ ID NO: 22 - protein, 6 aa, Bacillus thuringiensis
QGFSHR

SEQ ID NO: 23 - protein, 28 aa, Bacillus thuringiensis
MDNNPNINECIPYNCLSNPEVEVLGGER

SEQ ID NO: 24 - protein, 19 aa, Bacillus thuringiensis
ELTLTVLDIVSLFPNYDSR

SEQ ID NO: 25 - protein, 34 aa, Bacillus thuringiensis
RPFNIGINNQQLSVLDGTEFAYGTSSNLPSAVYR

SEQ ID NO: 26 - protein, 20 aa, Bacillus thuringiensis
SGTVDSLDEIPPQNNNVPPR

SEQ ID NO: 27 - protein, 11 aa, Artificial Sequence
  Engineered hybrid toxin
TDVTDYHIDQV

SEQ ID NO: 28 - protein, 15 aa, Artificial Sequence
  Engineered hybrid toxin
AVNELFTSSNQIGLK

SEQ ID NO: 29 - protein, 8 aa, Artificial Sequence
  Engineered hybrid toxin
ITQLPLTK

SEQ ID NO: 30 - protein, 8 aa, Artificial Sequence
  Engineered hybrid toxin
GLDSSTTK

SEQ ID NO: 31 - protein, 11 aa, Artificial Sequence
  Engineered hybrid toxin
QCAGIRPYDGR

SEQ ID NO: 32 - protein, 18 aa, Artificial Sequence
  Engineered hybrid toxin
IEFVPAEVTFEAEYDLER

SEQ ID NO: 33 - protein, 8 aa, Artificial Sequence
  Mutated Cry3A
ITQLPLVK

SEQ ID NO: 34 - protein, 16 aa, Artificial Sequence
  Mutated Cry3A
MTADNNTEALDSSTTK

SEQ ID NO: 35 - protein, 5 aa, Artificial Sequence
  Mutated Cry3A
VYIDK

SEQ ID NO: 36 - protein, 11 aa, Bacillus thuringiensis
DGGISQFIGDK

SEQ ID NO: 37 - protein, 7 aa, Bacillus thuringiensis
LITLTCK

SEQ ID NO: 38 - protein, 11 aa, Bacillus thuringiensis
ELLLATDLSNK

SEQ ID NO: 39 - protein, 13 aa, Bacillus thuringiensis
FEELTFATETSSK

SEQ ID NO: 40 - protein, 6 aa, Bacillus thuringiensis
EVLFEK

SEQ ID NO: 41 - protein, 8 aa, Bacillus thuringiensis
TASELITK

SEQ ID NO: 42 - protein, 9 aa, Bacillus thuringiensis
DVSEMFTTK

SEQ ID NO: 43 - protein, 19 aa, Bacillus thuringiensis
LLGLADIDYTSIMNEHLNK

SEQ ID NO: 44 - protein, 5 aa, Bacillus thuringiensis
IDFTK

SEQ ID NO: 45 - protein, 14 aa, Bacillus thuringiensis
TDTGGDLTLDEILK

SEQ ID NO: 46 - protein, 8 aa, Bacillus thuringiensis
DIMNMIFK

SEQ ID NO: 47 - protein, 6 aa, Bacillus thuringiensis
ALYVHK

SEQ ID NO: 48 - protein, 18 aa, Bacillus thuringiensis
VNILPTLSNTFSNPNYAK

SEQ ID NO: 49 - protein, 10 aa, Bacillus thuringiensis
ITSMLSDVIK

SEQ ID NO: 50 - protein, 12 aa, Bacillus thuringiensis
QNLQLDSFSTYR

SEQ ID NO: 51 - protein, 13 aa, Bacillus thuringiensis
DSLSEVIYGDMDK

SEQ ID NO: 52 - protein, 25 aa, Bacillus thuringiensis
MIVEAKPGHALIGFEISNDSITVLK

SEQ ID NO: 53 - protein, 12 aa, Bacillus thuringiensis
VYFSVSGDANVR

SEQ ID NO: 54 - protein, 11 aa, Bacillus thuringiensis
NQQLLNDISGK

SEQ ID NO: 55 - protein, 9 aa, Bacillus thuringiensis
VESSEAEYR

SEQ ID NO: 56 - protein, 6 aa, Bacillus thuringiensis
YMSGAK

SEQ ID NO: 57 - protein, 19 aa, Bacillus thuringiensis
DGSPADILDELTELTELAK

SEQ ID NO: 58 - protein, 5 aa, Bacillus thuringiensis
VYEAK

SEQ ID NO: 59 - protein, 9 aa, Bacillus thuringiensis
LDAINTMLR

SEQ ID NO: 60 - protein, 8 aa, Bacillus thuringiensis
GKPSIHLK

SEQ ID NO: 61 - protein, 24 aa, Bacillus thuringiensis
DENTGYIHYEDTNNNLEDYQTINK

SEQ ID NO: 62 - protein, 27 aa, Bacillus thuringiensis
DNFYIELSQGNNLYGGPIVHFYDVSIK

SEQ ID NO: 63 - protein, 26 aa, Bacillus thuringiensis
LLCPDQSEQIYYTNNIVFPNEYVITK

SEQ ID NO: 64 - protein, 22 aa, Bacillus thuringiensis
SQNGDEAWGDNFIILEISPSEK

SEQ ID NO: 65 - protein, 14 aa, Bacillus thuringiensis
NAYVDHTGGVNGTK

SEQ ID NO: 66 - protein, 23 aa, Bacillus thuringiensis
LDGVNGSLNDLIAQGNLNTELSK

SEQ ID NO: 67 - protein, 15 aa, Bacillus thuringiensis
IANEQNQVLNDVNNK

SEQ ID NO: 68 - protein, 19 aa, Bacillus thuringiensis
YEVTANFYDSSTGEIDLNK

SEQ ID NO: 69 - protein, 14 aa, Bacillus thuringiensis
QNYALSLQIEYLSK

SEQ ID NO: 70 - protein, 8 aa, Bacillus thuringiensis
QLQEISDK

SEQ ID NO: 71 - protein, 32 aa, Bacillus thuringiensis
LLSPELINTNNWTSTGSTNISGNTLTLYQGGR

SEQ ID NO: 72 - protein, 5 aa, Bacillus thuringiensis
YVNEK

SEQ ID NO: 73 - protein, 7 aa, Bacillus thuringiensis
QNYQVDK

SEQ ID NO: 74 - protein, 13 aa, Artificial Sequence
  Mutated EPSPS
MAGAEEIVLQPIK

SEQ ID NO: 75 - protein, 7 aa, Artificial Sequence
  Mutated EPSPS
FPVEDAK

SEQ ID NO: 76 - protein, 7 aa, Artificial Sequence
  Mutated EPSPS
EISGTVK

SEQ ID NO: 77 - protein, 31 aa, Artificial Sequence
  Mutated EPSPS
ILLLAALSEGTTVVDNLLNSEDVHYMLGALR

SEQ ID NO: 78 - protein, 17 aa, Streptomyces viridochromogenes
DFELPAPPRPVRPVTQI

SEQ ID NO: 79 - protein, 13 aa, Streptomyces viridochromogenes
LGLGSTLYTHLLK

SEQ ID NO: 80 - protein, 5 aa, Streptomyces viridochromogenes
MSPER

SEQ ID NO: 81 - protein, 12 aa, Streptomyces viridochromogenes
HGGWHDVGFWQR

SEQ ID NO: 82 - protein, 16 aa, Streptomyces viridochromogenes
NAYDWTVESTVYVSHR

SEQ ID NO: 83 - protein, 15 aa, Streptomyces viridochromogenes
TEPQTPQEWIDDLER

SEQ ID NO: 84 - protein, 5 aa, Streptomyces viridochromogenes
AAGYK

SEQ ID NO: 85 - protein, 22 aa, Streptomyces viridochromogenes
YPWLVAEVEGVVAGIAYAGPWK

SEQ ID NO: 86 - protein, 32 aa, Streptomyces viridochromogenes
RPVEIRPATAADMAAVCDIVNHYIETSTVNFR

SEQ ID NO: 87 - protein, 13 aa, Escherichia coli
ENAAGIPMDAAER

SEQ ID NO: 88 - protein, 6 aa, Escherichia coli
ALAILK

SEQ ID NO: 89 - protein, 15 aa, Escherichia coli
SALDSQQGEPWQTIR

SEQ ID NO: 90 - protein, 25 aa, Escherichia coli
GSQQLQLKPGESAFIAANESPVTVK

SEQ ID NO: 91 - protein, 15 aa, Escherichia coli
FEAKPANQLLTQPVK

SEQ ID NO: 92 - protein, 10 aa, Escherichia coli
STLLGEAVAK

SEQ ID NO: 93 - protein, 13 aa, Escherichia coli
LINSVQNYAWGSK

SEQ ID NO: 94 - protein, 9 aa, Escherichia coli
HNSEIGFAK

SEQ ID NO: 95 - protein, 16 aa, Escherichia coli
VLCAAQPLSIQVHPNK

SEQ ID NO: 96 - protein, 27 aa, Escherichia coli
TALTELYGMENPSSQPMAELWMGAHPK

SEQ ID NO: 97 - protein, 16 aa, Escherichia coli
LSELFASLLNMQGEEK

SEQ ID NO: 98 - protein, 24 aa, Escherichia coli
QGAELDFPIPVDDFAFSLHDLSDK

SEQ ID NO: 99 - protein, 7 aa, Bacillus thuringiensis
GIEGSIR

SEQ ID NO: 100 - protein, 5 aa, Bacillus thuringiensis
EGSIR

SEQ ID NO: 101 - protein, 9 aa, Bacillus thuringiensis
AQLGQGVYR

SEQ ID NO: 102 - protein, 6 aa, Bacillus thuringiensis
GQGVYR

SEQ ID NO: 103 - protein, 6 aa, Bacillus thuringiensis
SSTLYR

SEQ ID NO: 104 - protein, 5 aa, Bacillus thuringiensis
STLYR

SEQ ID NO: 105 - protein, 6 aa, Bacillus thuringiensis
SVFGQR

SEQ ID NO: 106 - protein, 4 aa, Bacillus thuringiensis
FGQR

SEQ ID NO: 107 - protein, 5 aa, Bacillus thuringiensis
SQLTR

SEQ ID NO: 108 - protein, 4 aa, Bacillus thuringiensis
QLTR

SEQ ID NO: 109 - protein, 6 aa, Bacillus thuringiensis
NTGLER

SEQ ID NO: 110 - protein, 7 aa, Bacillus thuringiensis
YNTGLER

SEQ ID NO: 111 - protein, 7 aa, Bacillus thuringiensis
PTNPALR

SEQ ID NO: 112 - protein, 8 aa, Bacillus thuringiensis
DPTNPALR

SEQ ID NO: 113 - protein, 5 aa, Bacillus thuringiensis
GPDSR

SEQ ID NO: 114 - protein, 5 aa, Bacillus thuringiensis
SWIHR

SEQ ID NO: 115 - protein, 6 aa, Bacillus thuringiensis
ATINSR

SEQ ID NO: 116 - protein, 8 aa, Bacillus thuringiensis
DAATINSR

SEQ ID NO: 117 - protein, 4 aa, Bacillus thuringiensis
AISR

SEQ ID NO: 118 - protein, 4 aa, Bacillus thuringiensis
EFAR

SEQ ID NO: 119 - protein, 5 aa, Bacillus thuringiensis
EEFAR

SEQ ID NO: 120 - protein, 9 aa, Bacillus thuringiensis
SNSSVSIIR

SEQ ID NO: 121 - protein, 7 aa, Bacillus thuringiensis
SSVSIIR

SEQ ID NO: 122 - protein, 4 aa, Bacillus thuringiensis
SMFR

SEQ ID NO: 123 - protein, 5 aa, Bacillus thuringiensis
VSMFR

SEQ ID NO: 124 - protein, 8 aa, Bacillus thuringiensis
ENFDGSFR

SEQ ID NO: 125 - protein, 4 aa, Bacillus thuringiensis
GSFR

SEQ ID NO: 126 - protein, 6 aa, Bacillus thuringiensis
YAESFR

SEQ ID NO: 127 - protein, 4 aa, Bacillus thuringiensis
NQFR

SEQ ID NO: 128 - protein, 4 aa, Bacillus thuringiensis
DLTR

SEQ ID NO: 129 - protein, 5 aa, Bacillus thuringiensis
NDLTR

SEQ ID NO: 130 - protein, 8 aa, Bacillus thuringiensis
TIYTDAHR

SEQ ID NO: 131 - protein, 6 aa, Bacillus thuringiensis
YTDAHR

SEQ ID NO: 132 - protein, 4 aa, Bacillus thuringiensis
PLTK

SEQ ID NO: 133 - protein, 8 aa, Bacillus thuringiensis
SAEFNNII

SEQ ID NO: 134 - protein, 4 aa, Bacillus thuringiensis
FSHR

SEQ ID NO: 135 - protein, 5 aa, Bacillus thuringiensis
GFSHR

SEQ ID NO: 136 - protein, 7 aa, Bacillus thuringiensis
EVLGGER

SEQ ID NO: 137 - protein, 4 aa, Bacillus thuringiensis
GGER

SEQ ID NO: 138 - protein, 7 aa, Bacillus thuringiensis
FPNYDSR

SEQ ID NO: 139 - protein, 6 aa, Bacillus thuringiensis
PNYDSR

SEQ ID NO: 140 - protein, 6 aa, Bacillus thuringiensis
PSAVYR

SEQ ID NO: 141 - protein, 9 aa, Bacillus thuringiensis
SGTVDSLDE

SEQ ID NO: 142 - protein, 8 aa, Artificial Sequence
  Engineered hybrid toxin
TDYHIDQV

SEQ ID NO: 143 - protein, 7 aa, Artificial Sequence
  Engineered hybrid toxin
DYHIDQV

SEQ ID NO: 144 - protein, 9 aa, Artificial Sequence
  Engineered hybrid toxin
TSSNQIGLK

SEQ ID NO: 145 - protein, 8 aa, Artificial Sequence
  Engineered hybrid toxin
SSNQIGLK

SEQ ID NO: 146 - protein, 6 aa, Artificial Sequence
  Engineered hybrid toxin
QLPLTK

SEQ ID NO: 147 - protein, 7 aa, Artificial Sequence
  Engineered hybrid toxin
TQLPLTK

SEQ ID NO: 148 - protein, 6 aa, Artificial Sequence
  Engineered hybrid toxin
DSSTTK

SEQ ID NO: 149 - protein, 5 aa, Artificial Sequence
  Engineered hybrid toxin
SSTTK

SEQ ID NO: 150 - protein, 5 aa, Artificial Sequence
  Engineered hybrid toxin
PYDGR

SEQ ID NO: 151 - protein, 6 aa, Artificial Sequence
  Mutated Cry3A
QLPLVK

SEQ ID NO: 152 - protein, 7 aa, Artificial Sequence
  Mutated Cry3A
TQLPLVK

SEQ ID NO: 153 - protein, 8 aa, Artificial Sequence
  Mutated Cry3A
ALDSSTTK

SEQ ID NO: 154 - protein, 9 aa, Artificial Sequence
  Mutated Cry3A
EALDSSTTK

SEQ ID NO: 155 - protein, 4 aa, Artificial Sequence
  Mutated Cry3A
YIDK

SEQ ID NO: 156 - protein, 7 aa, Bacillus thuringiensis
SQFIGDK

SEQ ID NO: 157 - protein, 5 aa, Bacillus thuringiensis
TLTCK

SEQ ID NO: 158 - protein, 7 aa, Bacillus thuringiensis
ATDLSNK

SEQ ID NO: 159 - protein, 8 aa, Bacillus thuringiensis
LATDLSNK

SEQ ID NO: 160 - protein, 9 aa, Bacillus thuringiensis
TFATETSSK

SEQ ID NO: 161 - protein, 8 aa, Bacillus thuringiensis
FATETSSK

SEQ ID NO: 162 - protein, 4 aa, Bacillus thuringiensis
LFEK

SEQ ID NO: 163 - protein, 6 aa, Bacillus thuringiensis
SELITK

SEQ ID NO: 164 - protein, 7 aa, Bacillus thuringiensis
ASELTIK

SEQ ID NO: 165 - protein, 7 aa, Bacillus thuringiensis
SEMFTTK

SEQ ID NO: 166 - protein, 7 aa, Bacillus thuringiensis
MNEHLNK

SEQ ID NO: 167 - protein, 7 aa, Bacillus thuringiensis
MNEHLNK

SEQ ID NO: 168 - protein, 4 aa, Bacillus thuringiensis
DFTK

SEQ ID NO: 169 - protein, 7 aa, Bacillus thuringiensis
TLDEILK

SEQ ID NO: 170 - protein, 8 aa, Bacillus thuringiensis
LTLDEILK

SEQ ID NO: 171 - protein, 6 aa, Bacillus thuringiensis
MNMIFK

SEQ ID NO: 172 - protein, 5 aa, Bacillus thuringiensis
NMIFK

SEQ ID NO: 173 - protein, 4 aa, Bacillus thuringiensis
YVHK

SEQ ID NO: 174 - protein, 4 aa, Bacillus thuringiensis
VNIL

SEQ ID NO: 175 - protein, 8 aa, Bacillus thuringiensis
SMLSDVIK

SEQ ID NO: 176 - protein, 9 aa, Bacillus thuringiensis
TSMLSDVIK

SEQ ID NO: 177 - protein, 7 aa, Bacillus thuringiensis
DSFSTYR

SEQ ID NO: 178 - protein, 8 aa, Bacillus thuringiensis
LDSFSTYR

SEQ ID NO: 179 - protein, 7 aa, Bacillus thuringiensis
IYGDMDK

SEQ ID NO: 180 - protein, 8 aa, Bacillus thuringiensis
VIYGDMDK

SEQ ID NO: 181 - protein, 9 aa, Bacillus thuringiensis
SNDSITVLK

SEQ ID NO: 182 - protein, 7 aa, Bacillus thuringiensis
SGDANVR

SEQ ID NO: 183 - protein, 9 aa, Bacillus thuringiensis
SVSGDANVR

SEQ ID NO: 184 - protein, 8 aa, Bacillus thuringiensis
LLNDISGK

SEQ ID NO: 185 - protein, 7 aa, Bacillus thuringiensis
LNDISGK

SEQ ID NO: 186 - protein, 7 aa, Bacillus thuringiensis
SSEAEYR

SEQ ID NO: 187 - protein, 8 aa, Bacillus thuringiensis
ESSEAEYR

SEQ ID NO: 188 - protein, 4 aa, Bacillus thuringiensis
SGAK

SEQ ID NO: 189 - protein, 5 aa, Bacillus thuringiensis
MSGAK

SEQ ID NO: 190 - protein, 8 aa, Bacillus thuringiensis
TELTELAK

SEQ ID NO: 191 - protein, 7 aa, Bacillus thuringiensis
DGSPADI

SEQ ID NO: 192 - protein, 4 aa, Bacillus thuringiensis
YEAK

SEQ ID NO: 193 - protein, 5 aa, Bacillus thuringiensis
NTMLR

SEQ ID NO: 194 - protein, 7 aa, Bacillus thuringiensis
AINTMLR

SEQ ID NO: 195 - protein, 6 aa, Bacillus thuringiensis
PSIHLK

SEQ ID NO: 196 - protein, 7 aa, Bacillus thuringiensis
DYQTINK

SEQ ID NO: 197 - protein, 4 aa, Bacillus thuringiensis
DNFY

SEQ ID NO: 198 - protein, 8 aa, Bacillus thuringiensis
PNEYVITK

SEQ ID NO: 199 - protein, 5 aa, Bacillus thuringiensis
SPSEK

SEQ ID NO: 200 - protein, 8 aa, Bacillus thuringiensis
LEISPSEK

SEQ ID NO: 201 - protein, 11 aa, Bacillus thuringiensis
DHTGGVNSGTK

SEQ ID NO: 202 - protein, 9 aa, Bacillus thuringiensis
GNLNTELSK

SEQ ID NO: 203 - protein, 6 aa, Bacillus thuringiensis
NTELSK

SEQ ID NO: 204 - protein, 7 aa, Bacillus thuringiensis
LNDVNNK

SEQ ID NO: 205 - protein, 6 aa, Bacillus thuringiensis
NDVNNK

SEQ ID NO: 206 - protein, 4 aa, Bacillus thuringiensis
DLNK

SEQ ID NO: 207 - protein, 7 aa, Bacillus thuringiensis
QIEYLSK

SEQ ID NO: 208 - protein, 8 aa, Bacillus thuringiensis
LQIEYLSK

SEQ ID NO: 209 - protein, 6 aa, Bacillus thuringiensis
QEISDK

SEQ ID NO: 210 - protein, 5 aa, Bacillus thuringiensis
YQGGR

SEQ ID NO: 211 - protein, 7 aa, Bacillus thuringiensis
TLYQGGR

SEQ ID NO: 212 - protein, 4 aa, Bacillus thuringiensis
VNEK

SEQ ID NO: 213 - protein, 8 aa, Artificial Sequence
  Mutated EPSPS
EIVLQPIK

SEQ ID NO: 214 - protein, 6 aa, Artificial Sequence
  Mutated EPSPS
PVEDAK

SEQ ID NO: 215 - protein, 5 aa, Artificial Sequence
  Mutated EPSPS
VEDAK

SEQ ID NO: 216 - protein, 5 aa, Artificial Sequence
  Mutated EPSPS
SGTVK

SEQ ID NO: 217 - protein, 4 aa, Artificial Sequence
  Mutated EPSPS
GTVK

SEQ ID NO: 218 - protein, 6 aa, Artificial Sequence
  Mutated EPSPS
ILLLAA

SEQ ID NO: 219 - protein, 8 aa, Artificial Sequence
  Mutated EPSPS
HYMLGALR

SEQ ID NO: 220 - protein, 6 aa, Streptomyces viridiochromes
YTHLLK

SEQ ID NO: 221 - protein, 5 aa, Streptomyces viridiochromes
THLLK

SEQ ID NO: 222 - protein, 4 aa, Streptomyces viridiochromes
SPER

SEQ ID NO: 223 - protein, 5 aa, Streptomyces viridiochromes
GFWQR

SEQ ID NO: 224 - protein, 6 aa, Streptomyces viridiochromes
VGFWQR

SEQ ID NO: 225 - protein, 8 aa, Streptomyces viridiochromes
STVYVSHR

SEQ ID NO: 226 - protein, 5 aa, Streptomyces viridiochromes
TEPQT

SEQ ID NO: 227 - protein, 4 aa, Streptomyces viridiochromes
DLER

SEQ ID NO: 228 - protein, 4 aa, Streptomyces viridiochromes
AGYK

SEQ ID NO: 229 - protein, 4 aa, Streptomyces viridiochromes
GPWK

SEQ ID NO: 230 - protein, 9 aa, Streptomyces viridiochromes
GIAYAGPWK

SEQ ID NO: 231 - protein, 7 aa, Streptomyces viridiochromes
TSTVNFR

SEQ ID NO: 232 - protein, 7 aa, Eschericia coli
PMDAAER

SEQ ID NO: 233 - protein, 9 aa, Eschericia coli
GIPMDAAER

SEQ ID NO: 234 - protein, 4 aa, Eschericia coli
AILK

SEQ ID NO: 235 - protein, 6 aa, Eschericia coli
PWQTIR

SEQ ID NO: 236 - protein, 8 aa, Eschericia coli
GEPWQTIR

SEQ ID NO: 237 - protein, 9 aa, Eschericia coli
ANESPVTVK

SEQ ID NO: 238 - protein, 5 aa, Eschericia coli
PVTVK

SEQ ID NO: 239 - protein, 6 aa, Eschericia coli
LTQPVK

SEQ ID NO: 240 - protein, 6 aa, Eschericia coli
GEAVAK

SEQ ID NO: 241 - protein, 7 aa, Eschericia coli
LGEAVAK

SEQ ID NO: 242 - protein, 8 aa, Eschericia coli
QNYAWGSK

SEQ ID NO: 243 - protein, 7 aa, Eschericia coli
NYAWGSK

SEQ ID NO: 244 - protein, 8 aa, Eschericia coli
NSEIGFAK

SEQ ID NO: 245 - protein, 6 aa, Eschericia coli
VLCAAQ

SEQ ID NO: 246 - protein, 7 aa, Eschericia coli
WMGAHPK

SEQ ID NO: 247 - protein, 5 aa, Eschericia coli
TALTE

SEQ ID NO: 248 - protein, 7 aa, Eschericia coli
NMQGEEK

SEQ ID NO: 249 - protein, 8 aa, Eschericia coli
LNMQGEEK

SEQ ID NO: 250 - protein, 8 aa, Eschericia coli
SLHDLSDK

SEQ ID NO: 251 - protein, 6 aa, Eschericia coli
HDLSDK

SEQ ID NO: 252 - protein, 12 aa, Artificial Sequence
  Mutated Cry3A
LQSGASVVAGPR

SEQ ID NO: 253 - protein, 10 aa, Artificial Sequence
  modified Cry3A peptide
SGASVVAGPR

SEQ ID NO: 254 - protein, 7 aa, Artificial Sequence
  modified Cry3A peptide
SVVAGPR

SEQ ID NO: 255 - protein, 8 aa, Bacillus thuringiensis
FTTGTDLK

SEQ ID NO: 256 - protein, 6 aa, Bacillus thuringiensis
TGTDLK

SEQ ID NO: 257 - protein, 22 aa, Artificial Sequence
  Mutated EPSPS
SLTAAVTAAGGNATYVLDGVPR

SEQ ID NO: 258 - protein, 4 aa, Artificial Sequence
  dmEPSPS transition 2
GVPR

SEQ ID NO: 259 - protein, 615 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEY

SEQ ID NO: 260 - protein, 653 aa, Artificial Sequence
  Engineered hybrid toxin
MTSNGRQCAGIRPYDGRQQHRGLDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQV

SEQ ID NO: 261 - protein, 598 aa, Artificial Sequence
  Mutated Cry3A
MTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPAAPFRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 262 - protein, 789 aa, Artificial Sequence
  Modified Vip3A
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 263 - protein, 445 aa, Artificial Sequence
  Double mutatnt 5-enolpyruvulshikimate-3- phosphate synthase
MAGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFLGNAGIAMRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 264 - protein, 191 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQIPDRSNIWQ

SEQ ID NO: 265 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 266 - protein, 391 aa, Artificial Sequence
  MIR604 PMI
MQKLINSVQNYAWGSKTALTELYGMENPSSQPMAELWMGAHPKSSSRVQNAAGDIVSLRDAIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQHGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 267 - protein, 881 aa, Artificial Sequence
  Engineered hybrid toxin
MDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 268 - protein, 605 aa, Bacillus thuringiensis
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLE

SEQ ID NO: 269 - protein, 123 aa, Bacillus thuringiensis
MSAREVHIDVNNKTGHTLQLEDKTKLDGGRWRTSPTNVANDQIKTFVAESNGFMTGTEGTIYYSINGEAEISLYFDNPFAGSNKYDGHSNKSQYEIITQGGSGNQSHVTYTIQTTSSRYGHKS

SEQ ID NO: 270 - protein, 354 aa, Bacillus thuringiensis
MLDTNKVYEISNHANGLYAATYLSLDDSGVSLMNKNDDDIDDYNLKWFLFPIDDDQYIITSYAANNCKVWNVNNDKINVSTYSSTNSIQKWQIKANGSSYVIQSDNGKVLTAGTGQALGLIRLTDESSNNPNQQWNLTSVQTIQLPQKPIIDTKLKDYPKYSPTGNIDNGTSPQLMGWTLVPCIMVNDPNIDKNTQIKTTPYYILKKYQYWQRAVGSNVALRPHEKKSYTYEWGTEIDQKTTIINTLGFQINIDSGMKFDIPEVGGGTDEIKTQLNEELKIEYSHETKIMEKYQEQSEIDNPTDQSMNSIGFLTITSLELYRYNGSEIRIMQIQTSDNDTYNVTSYPNHQQALL

(19) United States
(12) Patent Application Publication
(10) Pub. No.: US 2021/0190794 A1
(43) Pub. Date: Jun. 24, 2021

(54) COMPOSITIONS AND METHODS FOR PROTEIN DETECTION

(71) Applicant: Syngenta Participations AG, Basel (CH)

(72) Inventors: Scott Young, Research Triangle Park, NC (US); Richard Sessler, Research Triangle Park, NC (US); Gerson Graser, Research Triangle Park, NC (US); Rudolf Guilbaud, Montreal, Quebec (CA); Michael Schirm, Montreal, Quebec (CA); Maxim Isabelle, Montreal, Quebec (CA)

(73) Assignee: Syngenta Participations AG, Basel (CH)

(21) Appl. No.: 17/271,991

(22) PCT Filed: Aug. 14, 2019
(86) PCT No.: PCT/US19/46438

§ 371 Date: Feb. 26, 2021

Related U.S. Application Data
(60) Provisional application No. 62/723,164, filed on Aug. 27, 2018.

(51) Int. Cl. G01N 33/68

(52) U.S. Cl. CPC G01N 33/6848; G01N 2458/15; G01N 2333/91182; G01N 2333/91051; G01N 2333/99; G01N 2333/325

(57) ABSTRACT

The invention relates generally to peptide biomarkers with specific ionization characteristics to directly quantify one or more transgenic target proteins in biological samples, including transgenic plant samples, by liquid chromatography coupled tandem mass spectrometry multiple reaction monitoring (MRM). The peptide biomarkers in combination with MRM-based methods may be used to quantify a single transgenic target protein or multiple transgenic target proteins within a stacked transgenic crop, such as maize, utilizing selected peptide biomarkers either alone or in combination. The present disclosure allows for broad based, reliable quantitation in different biological matrices, including plant matrices. The peptide biomarkers of the invention can further be used as trait biomarkers to support identification and/or selection of specific transgenic Events. Also provided are different peptide biomarker combinations that can be used to perform the methods of the invention.

### REFERENCE TO SEQUENCE LISTING SUBMITTED ELECTRONICALLY

The official copy of the sequence listing is submitted electronically via EFS-Web as an ASCII formatted sequence listing with a file named “81319-US-L-ORG-NAT-1_SeqList_ST25.txt”, created on Aug. 7, 2018, and having a size of 94 kilobytes and is filed concurrently with the specification. The sequence listing contained in this ASCII formatted document is part of the specification and is herein incorporated by reference in its entirety.

### FIELD OF THE INVENTION

The present invention relates generally to the use of mass spectrometry to selectively detect, quantify, and characterize target transgenic proteins in complex biological samples.

### BACKGROUND

Transgenic crops consist of increasingly complex genetic modifications including multiple transgenes that confer different traits, also called “gene stacks” or “trait stacks.” For example, many transgenic corn products currently on the market contain within the same plant multiple insecticidal proteins for controlling a broad spectrum of insect pests, multiple proteins that confer on the plant tolerance to a wide spectrum of chemical herbicides and multiple proteins that are used as selectable markers during the plant transformation process. Many of the transgenic proteins used to control insect pests, for example the crystal endotoxins from Bacillus thuringiensis (called Cry proteins) may be structurally closely related and have similar overall amino acid sequence identity or contain motifs or domains with significant identity to each other. Many Cry proteins are active against lepidopteran or coleopteran insect pests. Examples of lepidopteran-active Cry proteins include Cry1A, Cry1B, Cry1C, Cry1D, Cry1E, Cry1F and Cry9. Examples of coleopteran-active Cry proteins include, Cry3A, Cry3B, Cry3C, Cry8, the binary Cry23-Cry37 and the binary Cry34-Cry35. Most individual Cry proteins are biologically active against a narrow spectrum of insect species within a given insect Order.

Many successful attempts to create hybrid Cry proteins with increased spectrums of activity have been disclosed in the literature. For example, the silk moth (Bombyx mori) specificity domain from a Cry1Aa protein was moved to a Cry1Ac protein, thus imparting a new insecticidal activity to the resulting Cry1Aa-Cry1Ac chimeric protein (Ge et al. 1989, PNAS 86: 4037 4041). Thompson et al. 1996 and 1997 (U.S. Pat. Nos. 5,527,883 and 5,593,881) replaced the protoxin tail region of a wild-type Cry1F protein and Cry1C protein with the protoxin tail region of a Cry1Ab protein to make a Cry1F-Cry1Ab hybrid Cry protein and a Cry1C-Cry1Ab hybrid Cry protein, both having improved expression in certain expression host cells. Bosch et al. 1998 (U.S. Pat. No. 5,736,131), created new lepidopteran-active proteins by substituting domain III of a Cry1Ea protein and a Cry1Ab protein with domain III of Cry1Ca protein thus producing a Cry1E-Cry1C hybrid Cry protein called G27 and a Cry1Ab-Cry1C hybrid Cry protein called H04, both of which have a broader spectrum of lepidopteran activity than the wild-type Cry protein parent molecules. Malvar et al. 2001 (U.S. Pat. No. 6,242,241) combined domain I of a Cry1Ac protein with domains II and III and the protoxin tail of a Cry1F protein to create a Cry1Ac-Cry1F hybrid Cry protein with broader insecticidal activity than the parental wild-type Cry proteins. Bogdanova et al. 2011 (U.S. Pat. No. 8,034,997) combined domains I and II of a Cry1Ab protein with domain III of a Cry1Fa protein and added a Cry1Ac protein protoxin tail to create a new lepidopteran-active hybrid Cry protein called Cry1A.105. And, Hart et al. 2012 (U.S. Pat. No. 8,309,516) combined domains I and II of a Cry3A protein and a modified Cry3A protein with domain III of a Cry1Ab protein and added a portion of a Cry1Ab protein protoxin tail to create a coleopteran-active hybrid Cry protein called FR8a (also called eCry3.1Ab). Most of the reported hybrid Cry proteins to date have used all or parts of the same classes of wild-type Cry proteins, such as Cry1Aa, Cry1Ab, Cry1Ac, Cry1C, Cry1F and Cry3A.

Several wild-type Cry proteins, for example Cry1Ab, Cry1Ac, Cry1C, Cry1F, Cry2A, Cry2Ba, Cry3A, Cry3B, Cry9C and Cry34-Cry35, as well as vegetative insecticidal proteins, such as Vip3A (See U.S. Pat. No. 5,877,012), have been expressed in transgenic crop plants, including corn, cotton, rice and soybean, some of which have been exploited commercially to control certain lepidopteran and coleopteran insect pests since as early as 1996. More recently, transgenic crop products, e.g. corn, containing engineered Cry proteins having one or more amino acids substituted, deleted or inserted, for example modified Cry3A (mCry3A; U.S. Pat. No. 7,230,167), and hybrid Cry proteins, for example, eCry3.1Ab and Cry1A.105 described above, have been introduced commercially.

The increasing use of recombinant DNA technology to produce transgenic plants for commercial and industrial use requires the development of diagnostic methods of analyzing transgenic plant lines. Such methods are needed to maintain transgenic plant varieties through successive generations of breeding, to monitor the presence of transgenic plants or plant parts in the environment or in biological samples derived from the transgenic plants, and to assist in the rapid creation and development of new transgenic plants with desirable or optimal phenotypes. Moreover, current guidelines for the safety assessment of transgenic plants from many countries' regulatory agencies requires characterization at the DNA and protein level to obtain and maintain regulatory approval. The increasing complexity of the genes and proteins stacked into a transgenic plant as described above make specific detection and quantitation of any one target protein within the complex mixture difficult, particularly when the stacked transgenic proteins are similar to each other, or similar to wild-type non-transgenic proteins in the environment, or similar to non-transgenic proteins endogenous to the transgenic plant.

Immunoassay, e.g. enzyme linked immunosorbent assay (ELISA), is the current preferred method in the agricultural industry for detection and quantification of proteins introduced through genetic modification of plants. The crucial component of an immunoassay is an antibody with specificity for the target protein (antigen). Immunoassays can be highly specific and samples often need only a simple preparation before being analyzed. Moreover, immunoassays can be used qualitatively or quantitatively over a wide range of concentrations. Typically, immunoassays require separate tests for each protein of interest. The antibodies can be polyclonal, raised in animals, or monoclonal, produced by cell cultures. By their nature, a mixture of polyclonal antibodies will have multiple recognition epitopes, which can increase sensitivity, but it is also likely to reduce specificity, as the chances of sequence and structural homology with other proteins increases with the number of different antibody paratopes present. Monoclonal antibodies offer some advantages over polyclonal antibodies because they express uniform affinity and specificity against a single epitope or antigenic determinant and can be produced in vast quantities. However, there are intrinsic properties of all antibodies that limit their use for more demanding applications, such as selective detection and quantitation of single transgenic proteins in complex mixtures of similar transgenic or endogenous proteins. In addition, both polyclonal and monoclonal antibodies may require further purification steps to enhance the sensitivity and reduce backgrounds in assays. In addition, ELISA systems are likely unable to detect subtle changes to a target protein that may have a dramatic effect on its physical and biological properties. For example, the antibody might not recognize a specific form of the protein or peptide that has been altered by post-translation modification such as phosphorylation or glycosylation, or conformationally obscured, or modified by partial degradation. Identification of such modifications is vital because changes in the physical and biological properties of these proteins may play an important role in their enzymatic, clinical or other biological activities. Such changes can limit the reliability and utility of ELISA-based quantification methods.

Currently, making a valid identification of a transgenic plant product containing a transgenic protein or quantitating a transgenic protein in a commercial crop product depends on the accuracy of the immunoassay. Development of a successful immunoassay depends on certain characteristics of the antigen used for development of the antibody, i.e. size, hydrophobicity and the tertiary structure of the antigen and the quality and accuracy of the antibody. The specificity of antibodies must be checked carefully to elucidate any cross-reactivity with similar substances, which might cause false positive results. A current problem in the industry is that many of the antibodies in commercially available tests kits do not differentiate between similar transgenic proteins in various products or transgenic proteins from wild-type proteins, making differential product identification and quantitation difficult or impossible. For example, with many current commercial transgenic crop products using one or more of the same wild-type Cry proteins, for example Cry1Ab, Cry1Ac, Cry1F and Cry3, and with the introduction of crops expressing hybrid Cry proteins made of whole or parts of the same wild-type Cry proteins that are already in transgenic crop products, there is a continuing need to develop new and improved diagnostic methods to be able to distinguish wild-type Cry proteins from each other and from a hybrid Cry protein containing all or portions of that same wild-type Cry protein when they are together in complex biological samples, such as samples from transgenic plants, transgenic plant parts or transgenic microorganisms.

Mass spectrometry (MS) provides an alternative platform that overcomes many limitations of ELISA for protein analysis. The field of MS-based analysis has resulted in an important advancement of targeted protein analysis, such as multiple reaction monitoring (MRM) by electrospray liquid chromatography coupled with tandem mass spectrometry (LC-MS/MS). The underlying concept is that proteins may be quantified by measuring their specific constituent peptides (surrogate peptides) following proteolytic digestion. The acquisition of data only for the selected peptides allows measurements with higher precision, sensitivity, and throughput. Protein quantitation by MRM-based measurements of surrogate peptides is the most rapidly growing application of MS in protein analysis. MRM-based protein assays offer two compelling advantages over immuno-based assays, the first being the ability to systematically configure a specific assay for essentially any protein without the use of an antibody. The second is the ability of targeted MS assays to perform multiplexed analysis of many peptides in a single analysis. In addition, MRM is a direct analysis where immune-based assays are indirect. Immuno-based assays rely on a binding assay comprised of a ligating reagent that can be immobilized on a solid phase along with a detection reagent that will bind specifically and use an enzyme to generate a signal that can be properly quantified.

Commercial transgenic crop products comprise stacks of insecticidal proteins, herbicide tolerance proteins and selectable marker proteins. With many such commercial transgenic crop products using one or more of the same wild-type insecticidal Cry proteins, for example Cry1Ab, Cry1F and Cry3, and with the introduction of crops expressing hybrid Cry insecticidal proteins made of whole or parts of the same wild-type Cry proteins that are already in transgenic crop products, for example, mCry3A, eCry3.1Ab and Cry1A.105, an MRM-based assay must be capable of differentiating these closely related transgenic target insecticidal proteins as well as the herbicide tolerance and selectable marker proteins. Thus, there is a continuing need to identify surrogate peptides that have all the biochemical properties necessary to function in an MRM-based assay and have an additional property that they are absolutely specific to target transgenic proteins that may have large portions of their amino acid sequences that overlap, i.e. one or more of surrogate peptide's transition states are capable of clearly, without interference, differentiating two closely related target proteins across multiple complex matrices. Such selective surrogate peptides and their transition states should be capable of distinguishing target transgenic proteins that are similar to each other, or similar to wild-type non-transgenic proteins in the environment, or similar to non-transgenic proteins endogenous to the transgenic plant.

### SUMMARY

The present invention provides labeled surrogate peptides and their respective transition ions that are useful in selectively detecting or quantifying target transgenic proteins that are in a complex biological matrix using mass spectrometry. The invention further provides methods and systems for selectively detecting or quantifying the target transgenic proteins in the complex biological matrix using the labeled surrogate peptides and transition ions.

In one aspect of the invention, internal standard peptide markers are designed through empirical analysis and in silico digestion analysis; synthesized chemically with a heavy amino acid residue or genetically by expressing a synthetic gene in the presence of stable isotope-labeled amino acid(s) or metabolic intermediates. In certain embodiments, the internal standards may be characterized individually by mass spectrometry (MS) analysis, including tandem mass spectrometry (MS/MS) analysis, more specifically, liquid chromatography-coupled tandem mass spectrometry analysis (LC-MS/MS). After characterization, pre-selected parameters of the peptides can be collected, such as mono isotopic mass of each peptide, its surrogate charge state, the surrogate m/z value, the m/z transition ions, and the ion type of each transition ion. Other considerations include optimizing peptide size, avoiding post-translational modifications, avoiding process induced modifications and avoiding high rates of missed protease cleavages.

An exemplary list of unique stable isotope-labeled (SIL) surrogate peptides is provided herein, which includes peptides comprising any one of SEQ ID NOs:1-98 or a combination thereof for selective detection or quantitation of transgenic proteins selected from the group consisting of the insecticidal proteins Cry1Ab, eCry3.1Ab, mCry3A and Vip3, the herbicide tolerance proteins dmEPSPS and PAT, and the plant transformation selectable marker protein PMI that may be comprised in plants having single transgenic events, breeding stacks of multiple events or molecular stacks of multiple target transgenic proteins. Each surrogate peptide sequence and transition ions for each peptide derived from the seven proteins are useful in a mass spectrometry-based multiple reaction monitoring (MRM) assay.

In another aspect of the invention, the labelled surrogate peptide selectively detects or quantitates a Cry1Ab protein and comprises an amino acid sequence of any one of SEQ ID NOs:1-26. In another aspect, the labelled surrogate peptide selectively detects or quantitates a Cry1Ab protein and produces a transition ion having an amino acid sequence selected from at least one of SEQ ID NOs:99-141 or the peptides PIR, TY, VW, HR, YR or PPR. In an embodiment of this aspect, the labelled surrogate peptide comprises the amino acid sequence SAEFNNIIPSSQITQIPLTK (SEQ ID NO:21) and produces a transition ion consisting of the amino acid sequence PLTK (SEQ ID NO:132) or SAEFNNII (SEQ ID NO:133).

In another aspect of the invention, the labelled surrogate peptide selectively detects or quantitates an eCry3.1Ab protein and comprises an amino acid sequence of any one of SEQ ID NOs:27-32. In another aspect of the invention, the labelled surrogate peptide selectively detects or quantitates an eCry3.1Ab protein and produces a transition ion having an amino acid sequence selected from at least one of SEQ ID NOs:142-150 or the peptides DGR, IEF or LER. In an embodiment of this aspect, the labelled surrogate peptide comprises the amino acid sequence TDVTDYHIDQV (SEQ ID NO:27) and produces a transition ion consisting of the amino acid sequence TDYHIDQV (SEQ ID NO:142) or DYHIDQV (SEQ ID NO:143).

In another aspect of the invention, the labelled surrogate peptide selectively detects or quantitates a mCry3A protein and comprises an amino acid sequence of any one of SEQ ID NOs:33-35. In another aspect of the invention, the labelled surrogate peptide selectively detects or quantitates a mCry3A protein and produces a transition ion having an amino acid sequence selected from at least one of SEQ ID NOs:151-155 or the peptide IDK. In an embodiment of this aspect, the labelled surrogate peptide comprises the amino acid sequence LQSGASVVAGPR (SEQ ID NO:252) and produces a transition ion consisting of the amino acid sequence SGASVVAGPR (SEQ ID NO:253) or SVVAGPR (SEQ ID NO:254).

In another aspect of the invention, the labelled surrogate peptide selectively detects or quantitates a Vip3 protein and comprises an amino acid sequence of any one of SEQ ID NOs:36-73. In another aspect of the invention, the labelled surrogate peptide selectively detects or quantitates a Vip3 protein and produces a transition ion having an amino acid sequence selected from at least one of SEQ ID NOs:156-212 or the peptides TCK, FEK, DVS, FTK, HK, VNI, MIV, EAK, HLK, NK, DNF, LLC, NAY, YE, SDK, NEK, DK or VDK. In an embodiment of this aspect, the labelled surrogate peptide comprises the amino acid sequence DGGISQFIGDK (SEQ ID NO:36) and produces a transition ion consisting of the amino acid sequence SQFIGDK (SEQ ID NO:156) or the amino acid sequence GDK.

In another aspect of the invention, the labelled surrogate peptide selectively detects or quantitates a dmEPSPS protein and comprises an amino acid sequence of any one of SEQ ID NOs:74-77. In another aspect of the invention, the labelled surrogate peptide selectively detects or quantitates a dmEPSPS protein and produces a transition ion having an amino acid sequence selected from at least one of SEQ ID NOs:213-219 or the peptide PIK. In an embodiment of this aspect, the labelled surrogate peptide comprises the amino acid sequence SLTAAVTAAGGNATYVLDGVPR (SEQ ID NO:257) and produces a transition ion consisting of the amino acid sequence GVPR (SEQ ID NO:258) or the amino acid sequence PR.

In another aspect of the invention, the labelled surrogate peptide selectively detects or quantitates a PAT protein and comprises an amino acid sequence of any one of SEQ ID NOs:78-86. In another aspect of the invention, the labelled surrogate peptide selectively detects or quantitates a PAT protein and produces a transition ion having an amino acid sequence selected from at least one of SEQ ID NOs:220-231 or the peptides DFE, DF, PER, SHR, GYK or NFR. In an embodiment of this aspect, the labelled surrogate peptide comprises the amino acid sequence LGLGSTLYTHLLK (SEQ ID NO:79) and produces a transition ion consisting of the amino acid sequence YTHLLK (SEQ ID NO:220) or THLLK (SEQ ID NO:221).

In another aspect of the invention, the labelled surrogate peptide selectively detects or quantitates a PMI protein and comprises an amino acid sequence of any one of SEQ ID NOs:87-98. In another aspect of the invention, the labelled surrogate peptide selectively detects or quantitates a PMI protein and produces a transition ion having an amino acid sequence selected from at least one of SEQ ID NOs:232-251 or the peptides LK, PVK, HN or PNK. In an embodiment of this aspect, the labelled surrogate peptide comprises the amino acid sequence SALDSQQGEPWQTIR (SEQ ID NO:89) and produces a transition ion consisting of the amino acid sequence PWQTIR (SEQ ID NO:235) or GEPWQTIR (SEQ ID NO:236).

In other aspects of the invention, the labelled surrogate peptides of the invention and their resulting transition ions selectively detect or quantitate a Cry1Ab protein comprising SEQ ID NO:259, or an eCry3.1Ab protein comprising SEQ ID NO:260, or a mCry3A protein comprising SEQ ID NO:261, or a Vip3 protein comprising SEQ ID NO:262, or a dmEPSPS protein comprising SEQ ID NO:263, or a PAT protein comprising SEQ ID NO:264, or a PMI protein SEQ ID NO:265 or SEQ ID NO:266.

In other aspects, the labelled surrogate peptide of the invention selectively detects or quantitates a target protein of the invention when the target protein is in a biological sample from a transgenic plant. In some embodiments of this aspect, the biological sample is from leaf tissue, seed, grain, pollen or root tissue of the transgenic plant.

In other aspects of the invention, the labelled surrogate peptides of the invention and their resulting transition ions selectively detect or quantitate a Cry1Ab protein from a corn plant comprising the transgenic event Bt11, or an eCry3.1Ab protein from a corn plant comprising the transgenic event 5307, or a mCry3A protein from a corn plant comprising the transgenic event MIR604, or a Vip3 protein from a corn plant comprising the transgenic event MIR162 or from a cotton plant comprising the transgenic event COT102, or a dmEPSPS protein from a corn plant comprising the transgenic event GA21, or a PAT protein from a corn plant comprising the transgenic event Bt11, DAS-59122, TC1507, DP4114 or T25, or a PMI protein from a corn plant comprising the transgenic event MIR162, MIR604, 5307 or 3272.

Many different combinations of surrogate peptides may be monitored and quantified simultaneously by MRM assay with one or more of the specific surrogate peptides from Cry1Ab, eCry3.1Ab, mCry3A, Vip3, dmEPSPS, PAT and/or PMI proteins, and therefore provide a means of measuring the total amount of each of those proteins in a given protein preparation obtained from a biological sample by mass spectrometry. These peptides in conjunction with MRM based assays have numerous applications including quantitative peptide/protein analysis for determining expression levels at different growth stages of a transgenic plant, determining expression levels in different transgenic plant tissues and organs, including but not limited to leaf tissue, seed and grain, pollen and root tissue, determining potential exposure levels for regulatory risk assessments, determining different levels of proteins in food processing, comparative, and generational studies. In the broadest sense these unique surrogate peptides for the seven proteins may be used in combination with the MRM assay for numerous applications including agricultural applications, bioequivalence testing, biomarker, diagnostic, discovery, food, environmental, therapeutic monitoring in all type of biological and non-biological matrices. In some aspects of the invention, an assay cassette is provided that comprises one or more labelled surrogate peptides of the invention comprising any of SEQ ID NOs:1-98, which allows for the simultaneous and selective detection or quantitation of any one or more target proteins of the invention.

The invention also provides methods for selectively detecting or quantitating transgenic target proteins within a complex biological matrix, such as a biological sample from a transgenic plant expressing the transgenic target proteins. Such a method includes obtaining a sample from the transgenic plant, for example a sample from a leaf, seed or grain, pollen or a root; extracting proteins from the plant sample; concentrating the target protein pool by reducing the amount of non-transgenic insoluble proteins in the extract; digesting the soluble proteins in the extract with a selected enzyme, for example trypsin, resulting in an extract comprising peptide fragments, wherein the peptide fragments include at least one surrogate peptide specific for each target transgenic protein; adding an assay cassette of SIL peptides that specifically detect target proteins, wherein each labeled surrogate peptide has the same amino acid sequence as each surrogate peptide of the target transgenic proteins, and wherein the number of labeled surrogate peptides that are added is equal to the number of target transgenic proteins in the mixture; concentrating the surrogate peptides and the labeled surrogate peptides by reducing the amount of non-surrogate peptides in the mixture; resolving the peptide fragment mixture using liquid chromatography; analyzing the peptide fragment mixture using mass spectrometry, wherein detection of a transition ion fragment of a labeled surrogate peptide is indicative of the presence of a target transgenic protein from which the surrogate peptide is derived; and optionally, calculating an amount of a target transgenic protein in the biological sample by comparing mass spectrometry signals generated from the transition ion fragment with mass spectrometry signals generated by a transition ion of a labeled surrogate peptide. The SIL surrogate peptides derived from the transgenic proteins of the invention each have unique transition ions during mass spectrometry-based multiple reaction monitoring (MRM) assay. As such these peptides will generate selective MS ions due to slight changes in collision energy resulting in different degrees of ionization. For example, triple quadrupole MS can be used to produce high m/z ions that are peptide specific. As a result the method of the invention can provide a selective advantage, reducing endogenous background, relative to the use of lower m/z intense ion markers that may be known in the art.

In some aspects of the invention, the target protein that is selectively detected or quantitated in the method of the invention is a Cry1Ab protein, an eCry3.1Ab protein, a mCry3A protein, a Vip3 protein, a double mutant 5-enolpyruvylshikimate-3-phosphate synthase (dmEPSPS) protein, a phosphinothricin acetyltransferase (PAT) protein or a phosphomannose isomerase (PMI) protein.

In other aspects of the invention, a labelled surrogate peptide that is useful in the method of the invention to detect or quantify a Cry1Ab protein, an eCry3.1Ab protein, a mCry3A protein, a Vip3 protein, a double mutant 5-enolpyruvylshikimate-3-phosphate synthase (dmEPSPS) protein, a phosphinothricin acetyltransferase (PAT) protein or a phosphomannose isomerase (PMI) protein comprises any one of SEQ ID NOs:1-98.

In another aspect of the method of the invention, the labelled surrogate peptide selectively detects or quantitates a Cry1Ab and comprises an amino acid sequence of any one of SEQ ID NOs:1-26. In another aspect, the labelled surrogate peptide selectively detects or quantitates a Cry1Ab and produces a transition ion having an amino acid sequence selected from at least one of SEQ ID NOs:99-141 or the peptides PIR, TY, VW, HR, YR or PPR. In an embodiment of this aspect, the labelled surrogate peptide comprises the amino acid sequence SAEFNNIIPSSQITQIPLTK (SEQ ID NO:21) and produces a transition ion consisting of the amino acid sequence PLTK (SEQ ID NO:132) or SAEFNNII (SEQ ID NO:133). In another aspect, the Cry1Ab target protein is quantitated in the biological sample by comparing mass spectrometry signals generated from a transition ion fragment consisting of the amino acid sequence PLTK (SEQ ID NO: 132).

In another aspect of the method of the invention, the labelled surrogate peptide selectively detects or quantitates an eCry3.1Ab protein and comprises an amino acid sequence of any one of SEQ ID NOs:27-32. In another aspect of the invention, the labelled surrogate peptide selectively detects or quantitates an eCry3.1Ab protein and produces a transition ion having an amino acid sequence selected from at least one of SEQ ID NOs:142-150 or the peptides DGR, IEF or LER. In an embodiment of this aspect, the labelled surrogate peptide comprises the amino acid sequence TDVTDYHIDQV (SEQ ID NO:27) and produces a transition ion consisting of the amino acid sequence TDYHIDQV (SEQ ID NO:142) or DYHIDQV (SEQ ID NO:143). In another aspect, the eCry3.1Ab target protein is quantitated in the biological sample by comparing mass spectrometry signals generated from a transition ion fragment consisting of the amino acid sequence TDYHIDQV (SEQ ID NO:142). In another embodiment of this aspect, the labelled surrogate peptide comprises the amino acid sequence AVFNELFTSSNQIGLK (SEQ ID NO:28) and produces a transition ion consisting of the amino acid sequence TSSNQIGLK (SEQ ID NO:144) or SSNQIGLK (SEQ ID NO:145). In another aspect, the eCry3.1Ab target protein is quantitated in the biological sample by comparing mass spectrometry signals generated from a transition ion fragment consisting of the amino acid sequence TSSNQIGLK (SEQ ID NO:144).

In another aspect of the method of the invention, the labelled surrogate peptide selectively detects or quantitates a mCry3A protein and comprises an amino acid sequence of any one of SEQ ID NOs:33-35. In another aspect of the method, the labelled surrogate peptide selectively detects or quantitates a mCry3A protein and produces a transition ion having an amino acid sequence selected from at least one of SEQ ID NOs:151-155 or the peptide IDK. In an embodiment of this aspect, the labelled surrogate peptide comprises the amino acid sequence LQSGASVVAGPR (SEQ ID NO:252) and produces a transition ion consisting of the amino acid sequence SGASVVAGPR (SEQ ID NO:253) or SVVAGPR (SEQ ID NO:254). In another aspect, the mCry3A target protein is quantitated in the biological sample by comparing mass spectrometry signals generated from a transition ion fragment consisting of the amino acid sequence SGASVVAGPR (SEQ ID NO:253).

In another aspect of the method of the invention, the labelled surrogate peptide selectively detects or quantitates an Vip3 protein and comprises an amino acid sequence of any one of SEQ ID NOs:36-73. In another aspect of the method, the labelled surrogate peptide selectively detects or quantitates a Vip3 protein and produces a transition ion having an amino acid sequence selected from at least one of SEQ ID NOs:156-212 or the peptides TCK, FEK, DVS, FTK, HK, VNI, MIV, EAK, HLK, NK, DNF, LLC, NAY, YE, SDK, NEK, DK or VDK. In an embodiment of this aspect, the labelled surrogate peptide comprises the amino acid sequence DGGISQFIGDK (SEQ ID NO:36) and produces a transition ion consisting of the amino acid sequence SQFIGDK (SEQ ID NO:156) or the amino acid sequence GDK. In another aspect, the Vip3 target protein is quantitated in the biological sample by comparing mass spectrometry signals generated from a transition ion fragment consisting of the amino acid sequence SQFIGDK (SEQ ID NO:156). In another embodiment of this aspect, the labelled surrogate peptide comprises the amino acid sequence FTTGTDLK (SEQ ID NO:255) and produces a transition ion consisting of the amino acid sequence TGTDLK (SEQ ID NO:256) or the amino acid sequence LK. In another aspect, the Vip3 target protein is quantitated in the biological sample by comparing mass spectrometry signals generated from a transition ion fragment consisting of the amino acid sequence TGTDLK (SEQ ID NO:256).

In another aspect of the method of the invention, the labelled surrogate peptide selectively detects or quantitates a dmEPSPS protein and comprises an amino acid sequence of any one of SEQ ID NOs:74-77. In another aspect of the method, the labelled surrogate peptide selectively detects or quantitates a dmEPSPS protein and produces a transition ion having an amino acid sequence selected from at least one of SEQ ID NOs:213-219. In an embodiment of this aspect, the labelled surrogate peptide comprises the amino acid sequence SLTAAVTAAGGNATYVLDGVPR (SEQ ID NO:257) and produces a transition ion consisting of the amino acid sequence GVPR (SEQ ID NO:258) or the amino acid sequence PR. In another aspect, the dmEPSPS target protein is quantitated in the biological sample by comparing mass spectrometry signals generated from a transition ion fragment consisting of the amino acid sequence PR.

In another aspect of the method of on the invention, the labelled surrogate peptide selectively detects or quantitates a PAT protein and comprises an amino acid sequence of any one of SEQ ID NOs:78-86. In another aspect of the invention, the labelled surrogate peptide selectively detects or quantitates a PAT protein and produces a transition ion having an amino acid sequence selected from at least one of SEQ ID NOs:220-231 or the peptides DFE, DF, PER, SHR, GYK or NFR. In an embodiment of this aspect, the labelled surrogate peptide comprises the amino acid sequence LGLGSTLYTHLLK (SEQ ID NO:79) and produces a transition ion consisting of the amino acid sequence YTHLLK (SEQ ID NO:220) or THLLK (SEQ ID NO:221). In another aspect, the dmEPSPS target protein is quantitated in the biological sample by comparing mass spectrometry signals generated from a transition ion fragment consisting of the amino acid sequence YTHLLK (SEQ ID NO:220).

In another aspect of the method of the invention, the labelled surrogate peptide selectively detects or quantitates a PMI protein and comprises an amino acid sequence of any one of SEQ ID NOs:87-98. In another aspect of the invention, the labelled surrogate peptide selectively detects or quantitates a PMI protein and produces a transition ion having an amino acid sequence selected from at least one of SEQ ID NOs:232-251 or the peptides LK, PVK, HN or PNK. In an embodiment of this aspect, the labelled surrogate peptide comprises the amino acid sequence SALDSQQGEPWQTIR (SEQ ID NO:89) and produces a transition ion consisting of the amino acid sequence PWQTIR (SEQ ID NO:235) or GEPWQTIR (SEQ ID NO:236).

The invention further provides a system for high-throughput detection or quantitation of transgenic target proteins. Such system comprises a cassette of pre-designed labelled surrogate peptides that are specific for the transgenic target proteins; and one or more mass spectrometers.

Various objects, features, aspects, and advantages of the present invention will become more apparent from the following detailed description of preferred embodiments of the invention, along with the accompanying drawings and sequence listing.

### BRIEF DESCRIPTION OF SEQUENCES

SEQ ID NOs:1-26 are amino acid sequences of stable isotope-labeled surrogate peptides for selective detection and quantitation of a transgenic Cry1Ab protein.

SEQ ID NOs:27-32 are amino acid sequences of stable isotope-labeled surrogate peptides for selective detection and quantitation of a transgenic eCry3.1Ab protein.

SEQ ID NOs:33-35 are amino acid sequences of stable isotope-labeled surrogate peptides for selective detection and quantitation of a transgenic mCry3A protein.

SEQ ID NOs:36-73 are amino acid sequences of stable isotope-labeled surrogate peptides for selective detection and quantitation of a transgenic Vip3 protein.

SEQ ID NOs:74-77 are amino acid sequences of stable isotope-labeled surrogate peptides for selective detection and quantitation of a transgenic dmEPSPS protein.

SEQ ID NOs:78-86 are amino acid sequences of stable isotope-labeled surrogate peptides for selective detection and quantitation of a transgenic PAT protein.

SEQ ID NOs:87-98 are amino acid sequences of stable isotope-labeled surrogate peptides for selective detection and quantitation of a transgenic PMI protein.

SEQ ID NOs:99-141 are amino acid sequences of transition ions of the SIL surrogate peptides of SEQ ID NOs:1-26.

SEQ ID NOs:142-150 are amino acid sequences of transition products of the SIL surrogate peptides of SEQ ID NOs:27-32.

SEQ ID NOs:151-155 are amino acid sequences of transition products of the SIL surrogate peptides of SEQ ID NOs:33-35.

SEQ ID NOs:156-212 are amino acid sequences of transition products of the SIL surrogate peptides of SEQ ID NOs:36-72.

SEQ ID NOs:213-219 are amino acid sequences of transition products of the SIL surrogate peptides of SEQ ID NOs:74-77.

SEQ ID NOs:220-231 are amino acid sequences of transition products of the SIL surrogate peptides of SEQ ID NOs:79-86.

SEQ ID NOs:232-251 are amino acid sequences of transition products of the SIL surrogate peptides of SEQ ID NOs:87-98.

SEQ ID NOs:252-254 are amino acid sequences of an SIL surrogate peptide and its transition products for selective detection and quantitation of a transgenic mCry3A protein.

SEQ ID NOs:255-256 are amino acid sequences of an SIL surrogate peptide and a transition product for selective detection and quantitation of a transgenic Vip3A protein.

SEQ ID NOs:257-258 are amino acid sequences of an SIL surrogate peptide and a transition product for selective detection and quantitation of a transgenic dmEPSPS protein.

SEQ ID NOs: 259-270 are amino acid sequences of exemplary target transgenic proteins of the invention.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. It is also to be understood that the terminology used herein is for the purpose of describing particular embodiments only, and is not intended to limit the scope of the present invention. General references related to the invention include: Alwine et al. (1977) Proc. Nat. Acad. Sci. 74:5350-54; Baldwin (2004) Mol. Cell. Proteomics 3(1):1-9; Can and Annan (1997) Overview of peptide and protein analysis by mass spectrometry. In: Current Protocols in Molecular Biology, edited by Ausubel, et al. New York: Wiley, p. 10.21.1-10.21.27; Chang et al. (2000) Plant Physiol. 122(2):295-317; Domon and Aebersold (2006) Science 312(5771):212-17; Nain et al. (2005) Plant Mol. Biol. Rep. 23:59-65; Patterson (1998) Protein identification and characterization by mass spectrometry. In: Current Protocols in Molecular Biology, edited by Ausubel, et al. New York: Wiley, p. 10.22.1-10.22.24; Paterson and Aebersold (1995) Electrophoresis 16: 1791-1814; Rajagopal and Ahern (2001) Science 294(5551):2571-73; Sesikeran and Vasanthi (2008) Asia Pac. J. Clin. Nutr. 17 Suppl. 1:241-44; and Toplak et al. (2004) Plant Mol. Biol. Rep. 22:237-50.

### Definitions

As used herein and in the appended claims, the singular forms “a,” “an,” and “the” can mean one or more than one. Thus, for example, reference to “a plant” can mean a single plant or multiple plants.

As used herein, “and/or” refers to and encompasses any and all possible combinations of one or more of the associated listed items, as well as the lack of combinations when interpreted in the alternative, “or.”

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of” (and grammatical variants) means that the scope of a claim is to be interpreted to encompass the specified materials or steps recited in the claim” and those that do not materially alter the basic and novel characteristic(s)” of the claimed invention. Thus, the term “consisting essentially of” when used in a claim of this invention is not intended to be interpreted to be equivalent to “comprising.”

The term “Cry protein” as used herein refers to an insecticidal protein that is a globular protein molecule which under native conditions accumulates as a protoxin in crystalline form during sporulation phase of a Bacillus sp., for example Bacillus thuringiensis, growth cycle. The terms “Cry toxin” and “delta-endotoxin” can be used interchangeably with the term “Cry protein.” Current nomenclature for Cry proteins and gene that encode the Cry proteins is based upon amino acid sequence homology (Crickmore et al. (1998) Microbiol. Mol. Biol. Rev. 62:807-813). In this art-recognized classification, each Cry protein is assigned a unique name incorporating a primary rank (an Arabic number), a secondary rank (an uppercase letter), a tertiary rank (a lowercase letter), and a quaternary rank (another Arabic number). For example, according to Crickmoe et al., two Cry proteins with <45% homology would be assigned a unique primary rank, e.g. Cry1 and Cry2. Two Cry proteins with >45% but <70% homology would receive the same primary rank but would be assigned a different secondary rank, e.g. Cry1A and Cry1B. Two Cry proteins with 70% to 95% homology would be assigned the same primary and secondary rank but would be assigned a different tertiary rank, e.g. Cry1Aa and Cry1Ab. And two Cry proteins with >95% but <100% homology would be assigned the same primary, secondary and tertiary rank, but would be assigned a different quaternary rank, e.g. Cry1Ab1 and Cry1Ab2.

A “Cry1Ab protein” as used herein means an insecticidal crystal protein derived from Bacillus thuringiensis, whether naturally occurring or synthetic, comprising an amino acid sequence that has at least 96% identity to the holotype Cry1Ab amino acid sequence according to Crickmore et al. (supra), and disclosed at the internet website “lifesci.sussex.ac.uk/home/Neil_Crickmore/Bt/” as Accession No. AAA22330. Examples of Cry1Ab proteins (with accession numbers) include without limitation, Cry1Ab1 (AAA22330), Cry1Ab2 (AAA22613), Cry1Ab3 (AAA22561), Cry1Ab4 (BAA00071), Cry1Ab5 (CAA28405), Cry1Ab6 (AAA22420), Cry1Ab7 (CAA31620), Cry1Ab8 (AAA22551), Cry1Ab9 (CAA38701), Cry1Ab10 (A29125), Cry1Ab11 (112419), Cry1Ab12 (AAC64003), Cry1Ab13 (AAN76494), Cry1Ab14 (AAG16877), Cry1Ab15 (AAO13302), Cry1Ab16 (AAK55546), Cry1Ab17 (AAT46415), Cry1Ab18(AAQ88259), Cry1Ab19 (AAW31761), Cry1Ab20 (ABB72460), Cry1Ab21 (ABS18384), Cry1Ab22 (ABW87320), Cry1Ab23 (HQ439777), Cry1Ab24 (HQ439778), Cry1Ab25 (HQ685122), Cry1Ab26 (HQ847729), Cry1Ab27 (JN135249), Cry1Ab28 (JN135250), Cry1Ab29 (JN135251), Cry1Ab30 (JN135252), Cry1Ab31 (JN135253), Cry1Ab32 (JN135254), Cry1Ab33 (AAS93798), Cry1Ab34 (KC156668), Cry1Ab35 (KT692985), and Cry1Ab36 (KY440260). An exemplary example of a Cry1Ab protein of the invention is represented by SEQ ID NO:259.

The term “Cry3” as used herein refers to insecticidal proteins that share a high degree of sequence identity or similarity to previously described sequences categorized as Cry3 according to Crickmore et al. (supra), examples of which are disclosed at the internet website “lifesci.sussex.ac.uk/home/Neil_Crickmore/Bt/” and include (with accession numbers), Cry3Aa1 (AAA22336), Cry3Aa2 (AAA22541), Cry3Aa3 (Caa68482), Cry3Aa4 (AAA22542), Cry3Aa5 (AAA50255), Cry3Aa6 (AAC43266), Cry3Aa7 (CAB41411), Cry3Aa8 (AAS79487), Cry3Aa9 (AAW05659), Cry3Aa10 (AAU29411), Cry3Aa11 (AAW82872), Cry3Aa12 (ABY49136), Cry3Ba1 (CAA34983), Cry3Ba2 (CAA00645), Cry3Ba3 (JQ397327), Cry3Bb1 (AAA22334), Cry3Bb2 (AAA74198), Cry3Bb3 (115475), and Cry3Ca1 (CAA42469). A Cry3 protein that has been engineered by inserting, substituting or deleting amino acids is referred to herein as a “modified Cry3 protein” or “mCry3 protein.” Such “modified Cry3 proteins” typically have enhanced activity against certain insect pests, e.g. corn rootworm (Diabrotica sp.), compared to a wild-type Cry3 protein from which the “modified Cry3 protein” is derived. An example of a “modified Cry3 protein” is the “mCry3A” represented by the amino acid sequence of SEQ ID NO:262. Other examples of “modified Cry3” proteins include without limitation the “mCry3A proteins” disclosed in U.S. Pat. No. 8,247,369, the “mCry3A proteins” disclosed in U.S. Pat. No. 9,109,231, and the “mCry3B proteins” disclosed in U.S. Pat. No. 6,060,594.

The term “eCry3.1Ab” refers to an engineered hybrid insecticidal protein comprising in an N-terminus to C-terminus direction an N-terminal region of a Cry3A protein fused to a C-terminal region of a Cry1Aa or a Cry1Ab protein as described in U.S. Pat. No. 8,309,516. An example of an “eCry3.1Ab protein” is represented by the amino acid sequence of SEQ ID NO:260.

As used herein the term transgenic “event” refers to a recombinant plant produced by transformation and regeneration of a single plant cell with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. Non-limiting examples of such transgenic events of the invention include “event Bt11,” comprising cry1Ab and pat genes and described in U.S. Pat. No. 6,114,608 (also “Bt11 event” or just “Bt11”), “event 5307,” comprising eCry3.1Ab and PMI genes and described in U.S. Pat. No. 8,466,346 (also “5307 event” or just “5307”), “event MIR604,” comprising mCry3A and PMI genes and described in U.S. Pat. No. 7,361,813 (also “MIR604 event” or just “MIR604”), “event MIR162,” comprising Vip3A and PMI genes and described in U.S. Pat. No. 8,232,456 (also “event MIR162” or just “MIR162”), “event GA21,” comprising a dmEPSPS gene and described in U.S. Pat. No. 6,566,587 (also “GA21 event” or just “GA21”), “event 3272,” comprising alpha-amylase797E and PMI genes and described in U.S. Pat. No. 7,635,799 (also “3272 event” or just “3272”), “event MON810,” comprising Cry1Ab and described in U.S. Pat. No. 6,713,259 (also “MON810 event” or just “MON810”), “event MON89034,” comprising Cry1A.105 and Cry2Ab genes and described in U.S. Pat. No. 8,062,840 (also “MON89034 event” or just “MON89034”), “event TC1507,” comprising Cry1F and PAT genes and described in U.S. Pat. No. 7,288,643 (also “TC1507 event” or just “TC1507”), “event DAS59122,” comprising Cry34/Cry35 and PAT genes and described in U.S. Pat. No. 7,323,556 (also “DAS59122 event” or just “DAS59122”) and “event DP4114,” comprising Cry1F, Cry34/Cry35 and PAT genes and described in U.S. Pat. No. 9,790,561 (also “DP4114 event” or just “DP4114”).

As used herein the term “hybrid Cry protein” is an engineered insecticidal protein that does not exist in nature and at least a portion of which comprises at least a contiguous 27% of a Cry1Ab protein's amino acid sequence. The 27% limitation is calculated by dividing the number of contiguous Cry1Ab amino acids in the hybrid Cry protein divided by the total number of amino acids in the hybrid Cry protein. For example, the hybrid Cry protein, eCry3.1Ab (SEQ ID NO:261) has 174 Cry1Ab amino acids (positions 480-653) and a total of 653 amino acids. Therefore, eCry3A.1Ab has at least a contiguous 27% of a Cry1Ab protein's amino acid sequence. Another example of a hybrid Cry protein, Cry1A.105, according to the present invention is represented by SEQ ID NO:267.

A “dmEPSPS” (5-enolpyruvulshikimate-3-phosphate synthase) is an engineered protein that confers onto a plant tolerance to a glyphosate herbicide as described in PCT publication No. WO97/04103. An exemplary example of a dmEPSPS of the invention is represented by SEQ ID NO:263.

“Highly related insecticidal proteins” as used herein refers to proteins that have at least 95% overall sequence identity or that have motifs in common that have at least 80% sequence identity. Examples of insecticidal proteins that are “highly related” include Cry1Ab (SEQ ID NO:259) and eCry3.1Ab (SEQ ID NO:260), that have a motif in common that has at least 80% sequence identity, and eCry3.1Ab (SEQ ID NO:260) and mCry3A (SEQ ID NO:261) that have a motif in common that has at least 80% sequence identity.

The term “isolated” nucleic acid molecule, polynucleotide or toxin is a nucleic acid molecule, polynucleotide or toxic protein that no longer exists in its natural environment. An isolated nucleic acid molecule, polynucleotide or toxin of the invention may exist in a purified form or may exist in a recombinant host such as in a transgenic bacterial cell or a transgenic plant.

As used herein, the general term “mass spectrometry” refers to any suitable mass spectrometry method, device or configuration including, e.g., electrospray ionization (ESI), matrix-assisted laser desorption/ionization (MALDI) MS, MALDI-time of flight (TOF) MS, atmospheric pressure (AP) MALDI MS, vacuum MALDI MS, tandem MS, or any combination thereof. Mass spectrometry devices measure the molecular mass of a molecule (as a function of the molecule's mass-to-charge ratio) by measuring the molecule's flight path through a set of magnetic and electric fields. The mass-to-charge ratio is a physical quantity that is widely used in the electrodynamics of charged particles. The mass-to-charge ratio of a particular peptide can be calculated, a priori, by one skilled in the art. Two particles with different mass-to-charge ratio will not move in the same path in a vacuum when subjected to the same electric and magnetic fields. The present invention includes, inter alia, the use of high performance liquid chromatography (HPLC) followed by tandem MS analysis of the peptides. In “tandem mass spectrometry,” a surrogate peptide may be filtered in an MS instrument, and the surrogate peptide subsequently fragmented to yield one or more “transition ions” that are analyzed (detected and/or quantitated) in a second MS procedure.

A detailed overview of mass spectrometry methodologies and devices can be found in the following references which are hereby incorporated by reference: Can and Annan (1997) Overview of peptide and protein analysis by mass spectrometry. In: Current Protocols in Molecular Biology, edited by Ausubel, et al. New York: Wiley, p. 10.21.1-10.21.27; Paterson and Aebersold (1995) Electrophoresis 16: 1791-1814; Patterson (1998) Protein identification and characterization by mass spectrometry. In: Current Protocols in Molecular Biology, edited by Ausubel, et al. New York: Wiley, p. 10.22.1-10.22.24; and Domon and Aebersold (2006) Science 312(5771):212-17.

A peptide is a short polymer formed from the linking, in a defined order, of alpha-amino acids. Peptides may also be generated by the digestion of polypeptides, for example proteins, with a protease.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “surrogate peptide” refers to a peptide that is derived from a target transgenic protein via proteolytic digestion, e.g. trypsin digestion, that functions in a mass spectrometry assay to produce one or more transition ions that in combination with the surrogate peptide differentially detects and/or quantitates the target transgenic protein when the target transgenic protein is in the presence of one or more other transgenic proteins and/or non-transgenic proteins in a complex biological matrix, such as a sample from a transgenic plant, and does not detect and/or quantitate the one or more other transgenic proteins or the non-transgenic proteins in the biological matrix. A “surrogate peptide” may also be referred to as a “signature peptide” for the target transgenic protein. For example, a Cry1Ab surrogate peptide of the invention produces one or more transition ions that in combination with a Cry1Ab-surrogate peptide differentially detects and/or quantitates a target Cry1Ab transgenic insecticidal protein in a complex biological matrix when the Cry1Ab transgenic protein is in the presence of one or more non-Cry1Ab transgenic proteins, for example, an eCry3.1Ab insecticidal protein or a mCry3A insecticidal protein of the invention, and/or non-transgenic proteins. In another example, an eCry3.1Ab surrogate peptide of the invention produces one or more transition ions that combined with a eCry3.1Ab-surrogate peptide differentially detects and/or quantitates a target eCry3.1Ab transgenic protein in a complex biological matrix when the eCry3.1Ab transgenic protein is in the presence of one or more non-eCry3.1Ab transgenic proteins, for example, Cry1Ab or mCry3A of the invention, and/or non-transgenic proteins in the complex biological matrix. According to embodiments of the invention, two or more labelled surrogate peptides of the invention may be used simultaneously in a mass spectrometry assay to detect and/or quantitate two or more target transgenic proteins in a complex biological matrix.

A “labeled surrogate peptide” is a non-naturally occurring surrogate peptide that is labeled for ease of detecting the surrogate peptide in a mass spectrometry assay. For example, the label can be a stable isotope labeled amino acid (SIL) such a lysine, isoleucine, valine or arginine. Thus, an SIL-labeled surrogate peptide has the same amino acid sequence as a non-labeled surrogate peptide except that one or more of the amino acids of the surrogate peptide are labeled with a heavy isotope. For example, as described herein, the surrogate peptide SAEFNNIIPSSQITQIPLTK (SEQ ID NO:21) is labeled with a heavy lysine (K) and may be designated SAEFNNIIPSSQITQIPLTK [C13N15-K]; the surrogate peptide TDVTDYHIDQV (SEQ ID NO:27) is labeled with a heavy valine (V) and may be designated as TDVTDYHIDQV[C13N15-V]; the surrogate peptide LQSGASVVAGPR (SEQ ID NO:252) is labeled with an arginine (R) and may be designated as LQSGASVVAGPR[C13N15-R]; the surrogate peptide DGGISQFIGDK (SEQ ID NO:36) is labeled with a heavy lysine (K) and may be designated as DGGISQFIGDK[C13N15-K]; the surrogate peptide FTTGTDLK (SEQ ID NO:255) is labeled with a heavy lysine (K) and may be designated as FTTGTDLK[C13N15-K]; the surrogate peptide SLTAAVTAAGGNATYVLDDGVPR (SEQ ID NO:257) is labeled with a heavy arginine (R) and may be designated as SLTAAVTAAGGNATYVLDDGVPR[C13N15-R]; the surrogate peptide LGLGSTLYTHLLK (SEQ ID NO:79) is labeled with a heavy lysine and may be designated as LGLGSTLYTHLLK[C13N15-K]; the surrogate peptide SALDSQQGEPWQTIR (SEQ ID NO:89) is labeled with a heavy arginine (R) and may be designated as SALDSQQGEPWQTIR[C13N15-R], and so on.

A “PAT” (phosphinothricin N-acetyltransferase) protein confers onto a plant tolerance to a glufosinate herbicide as described in PCT publication No. WO87/05629. An exemplary example of a PAT protein of the invention is represented by SEQ ID NO:264.

A “PMI” (mannose6-phosphate isomerase) protein confers upon a plant cell the ability to utilize mannose as described in U.S. Pat. No. 5,767,378. Exemplary examples of a PMI protein of the invention is represented by SEQ ID NO:265 and SEQ ID NO:266.

As used herein, the term “stacked” or “stacking” refers to the presence of multiple heterologous polynucleotides or transgenic proteins or transgenic events incorporated in the genome of a plant.

A “target protein” as used herein means a protein, typically a transgenic protein, which is intended to be selectively detected and/or quantitated by a labelled surrogate peptide when the target protein is in a complex biological matrix.

As used herein, the term “transgenic protein” means a protein or peptide produced in a non-natural form, location, organism, and the like. Therefore, a “transgenic protein” may be a protein with an amino acid sequence identical to a naturally-occurring protein or it may be a protein having a non-naturally occurring amino acid sequence. For example, a Cry1Ab protein having an amino acid sequence that is identical to a wild-type Cry1Ab protein from Bacillus thuringiensis, the native Cry1Ab-producing organism, is a “transgenic protein” when produced within a transgenic plant or bacteria.

Nucleotides are indicated herein by the following standard abbreviations: adenine (A), cytosine (C), thymine (T), and guanine (G). Amino acids are likewise indicated by the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The present invention encompasses compositions, methods and systems useful in carrying out mass spectrometry for differential detection and/or quantitation of one or more target transgenic proteins in complex biological samples derived from transgenic plants comprising a mixture of transgenic and non-transgenic proteins, for example, biological samples from leaves, stems, roots, pollen and seeds of one or more transgenic plants, each of which may impact mass spectrometry assay results differently. The compositions, methods and systems of the present invention are also useful for testing non-transgenic plants that are at risk of being contaminated with transgenes from neighboring plants, for example, by cross-pollination. By these embodiments, adventitious presence of transgenes may be monitored and confined. In other embodiments, methods disclosed herein may be used to screen the results of a plant transformation procedure to identify transformants that exhibit desirable expression characteristics of transgenic proteins.

Preference for the particular target proteins to be analyzed is at the discretion of the skilled artisan. Such proteins may be, but are not limited to, those from plants, animals, bacteria, yeast, and the like and may be proteins either not found in a non-transformed cell or found in a transformed cell. Particularly suitable proteins that are expressed in transgenic plants are those that confer tolerance to herbicides, insects, or viruses, and genes that provide improved nutritional value, increased yields, drought tolerance, nitrogen utilization, production of useful industrial compounds, processing characteristics of the plant, or potential for bioremediation. Examples of such proteins include the insecticidal crystal proteins, i.e. Cry proteins and vegetative insecticidal proteins, i.e. Vips, from Bacillus thuringiensis, or engineered proteins derived therefrom, for conferring insect resistance, herbicide tolerance proteins, such as 5′-enolpyruvyl-3′-phosphoshikimate synthase (EPSPS) or phosphinothricin acetyltransferase (PAT), or a selectable marker protein, such as phosphomannose isomerase (PMI). As is readily understood by those skilled in the art, any protein conferring a desired trait may be expressed in a plant cell using recombinant DNA technology and therefore may be a target transgenic protein according to the invention.

More particularly, the present invention provides compositions, diagnostic methods and systems useful in carrying out the diagnostic methods that allow for the specific differential detection and/or quantitation of Cry1Ab, eCry3.1Ab, mCry3A, Vip3, dmEPSPS, PAT and PMI transgenic proteins in complex biological matrices from samples of transgenic plant tissues such as leaves, roots, stems, pollen, seeds or grain. The compositions, diagnostic methods and systems of the invention are particularly useful for the differential detection and/or quantitation of highly similar transgenic insecticidal proteins, for example Cry1Ab, mCry3A and eCry3.1Ab, in complex biological samples comprising the transgenic insecticidal proteins. The current state of the art is such that commercially available immunoassays based on antibodies are not useful in differentially detecting a Cry1Ab protein from a hybrid Cry protein engineered using a significant amount of the Cry1Ab protein's amino acid sequence when the two proteins are in the same biological sample because there is high cross-reactivity of the antibodies between the two types of proteins. For example, an antibody raised against a wild-type Cry1Ab for use in a Cry1Ab-detecting immunoassay cross reacts with a hybrid Cry protein having as little as 27% of its amino acids derived from the wild-type Cry1Ab protein when the two proteins are in the same biological sample. Therefore, for example, the quantitation of the wild-type Cry1Ab in such a complex biological sample may be confounded by the presence of one or more non-target wild-type Cry proteins or non-target hybrid Cry proteins. Furthermore, using detection of expressed proteins for identity preservation of commercial transgenic plant products comprising a wild-type Cry1Ab and one or more hybrid Cry proteins of the present invention is difficult because of cross-reactivity of antibodies to both the Cry1Ab proteins and the hybrid Cry proteins in the transgenic plant products. The methods and compositions disclosed herein provide a solution to these problems and rely on surrogate peptides from the target transgenic proteins and transition ions derived from the surrogate peptides for the differential detection and/or quantitation of the target protein, even when the target protein is in a mixture of other very closely related transgenic proteins and non-transgenic proteins.

The accuracy of target protein quantitation by a mass spectrometry multiple reaction monitoring assay (MRM) is completely dependent on the selection of an appropriate surrogate peptide and on the target protein differentiating capability of the surrogate peptide/transition ion combination. Many different combinations of surrogate peptides of the invention may be monitored and quantified simultaneously by an MRM assay with one or more of the specific peptides from Cry1Ab, eCry3.1Ab, mCry3A, Vip3, dmEPSPS, PAT and/or PMI proteins, and therefore provide a means of identifying and quantifying each of the target proteins within a given biological sample by mass spectrometry. Surrogate peptides of the seven target proteins may make up a cassette to quantify each corresponding target protein, i.e. Cry1Ab, eCry3.1Ab, mCry3A, Vip3, dmEPSPS, PAT and/or PMI. The available surrogate peptides that make up the cassette may be analyzed alone or in any combination in a single MRM assay or analyzed in multiple MRM assays.

The surrogate peptides of the invention in conjunction with MRM based assays have numerous applications including quantitative peptide/protein analysis for determining expression levels at different growth stages, determining potential exposure levels for environmental risk assessments, determining different levels of target proteins in food processing, determining expression levels in comparative studies, and comparing expression levels in generational studies. In the broadest sense these unique surrogate peptides for the seven proteins may be used in combination with the MRM assay for monitoring or quantifying either selectable markers, herbicidal tolerance or insecticidal traits that may be in either single transgenic events, or breeding stacks of multiple transgenic events within a specific tissue (i.e. leaf, root, kernel, pollen).

The MRM based assays may either quantify or measure relative or absolute levels of specific surrogate peptides from proteins including Cry1Ab, eCry3.1Ab, mCry3A, Vip3, dmEPSPS, PAT and/or PMI. Relative quantitative levels of these proteins can be determined by the MRM assay by comparing signature peak areas to one another. The relative levels of individual Cry1Ab, eCry3.1Ab, mCry3A, Vip3, dmEPSPS, PAT and/or PMI surrogate peptides can be quantified from different samples or tissue types. In general, relative quantitative levels are determined by comparing peptide abundances in MRM measurements with a stable isotope-labeled (SIL) synthetic peptide analogue as an internal standard for each target surrogate peptide. Contrary to what is typically taught in the art, Typically, SIL peptides are labeled by incorporation of [¹³C₆¹⁵N₂] lysine or [¹³C₆¹⁵N₄] arginine, but may also include other amino acids such as isoleucine and valine. The SIL standard needs to be of high purity and should be quantitatively standardized by amino acid analysis. Contrary to what is typically taught in the art, the SIL's of the present invention are spiked into samples immediately after protein digestion and thus serve to correct for subsequent analytical steps. The SIL's co-elute with the unlabeled surrogate peptides in liquid chromatography separations and display identical MS/MS fragmentation patterns but differ only in mass due to the isotope labeling. This resulting mass shift in both labelled surrogate peptides and product ions allows the mass spectrometer to differentiate the unlabeled and labeled peptides. Because complex peptide digests often contain multiple sets of co-eluting transitions that may be mistaken for the target peptide, co-elution of the isotopically labeled standard identifies the correct signal and provides the best protection against false positive quantitation. Since a known concentration of a spiked SIL standard is spiked into each sample the relative quantitative amount of each corresponding surrogate peptide from the different target proteins may be determined for Cry1Ab, eCry3.1Ab, mCry3A, Vip3, dmEPSPS, PAT and/or PMI. Since relative quantitation of an individual peptide, or peptides, may be conducted relative to the amount of another peptide, or peptides, within or between samples, it is possible to determine the relative amounts of the peptides present by determining if the peak areas are relative to one another within the biological sample. Relative quantitative data derived from individual signature peak areas between different samples are generally normalized to the amount of protein analyzed per sample. Relative quantitation can be performed across many peptides from multiple proteins simultaneously in a single sample and/or across many samples to gain further insight into relative protein amounts, one peptide/protein with respect to other peptides/proteins.

Absolute quantitative levels may be determined for Cry1Ab, eCry3.1Ab, mCry3A, Vip3, mEPSPS, PAT and/or PMI by MRM based assays by comparing the signature peak area of an individual surrogate peptide from the corresponding proteins in one biological sample to a known amount of one or more internal standards in the sample. This may be achieved by spiking known concentrations of these proteins into negative control matrices which do not contain the target proteins. The multiple-reaction monitoring (MRM) assay comprises of weighing the non-transgenic sample with exact spiked concentrations of each of the seven proteins; extracting and homogenizing samples in a lysis buffer; centrifuging samples to separate soluble and insoluble proteins to enrich and reduce the complexity of the extraction; digesting soluble protein samples with trypsin (the tissue or biological sample may be treated with one or more proteases, including but not limited to trypsin, chymotrypsin, pepsin, endoproteinase Asp-N and Lys-C for a time to adequately digest the sample), centrifuging samples, adding a fixed concentration SIL peptide (in absolute quantitation the SIL is used as an indicator); desalting by solid-phase extraction utilizing cation exchange to minimize matrix effects or interferences and reduce ion suppression; and analyzing the sample by liquid chromatography coupled to tandem mass spectrometry. Typically an ion trap mass spectrometer, or another form of a mass spectrometer that is capable of performing global profiling, for identification of as many peptides as possible from a single complex protein/peptide lysate is typically performed for analysis. Although MRM-based assays can be developed and performed on any type of mass spectrometer, the most advantageous instrument platform for MRM assays is often considered to be a triple quadrupole instrument platform. The surrogate peptides of interest and SIL that are unique to the seven proteins are measured by LC-MS/MS. The peak area ratio (peak area of surrogate peptide/peak area of corresponding SIL peptide) is determined for each peptide of interest. The concentration of the seven proteins of interest is back-calculated from the calibration curve using the peak area ratio. Absolute quantitation can be performed across many peptides, which permits a quantitative determination of multiple proteins simultaneously in a single sample and/or across multiple samples to gain insight into absolute protein amounts in individual biological samples or large samples sets.

In some embodiments, the invention encompasses a labeled surrogate peptide that functions in a mass spectrometry assay, e.g. a multiple reaction monitoring assay, to selectively detect or quantitate a target transgenic protein selected from the group consisting of a Cry1Ab protein, an eCry3.1Ab protein, a mCry3A protein, a Vip3 protein, a double mutant 5-enolpyruvylshikimate-3-phosphate synthase (dmEPSPS) protein, a phosphinothricin acetyltransferase (PAT) protein and a phosphomannose isomerase (PMI) protein in a mixture of transgenic proteins and non-transgenic proteins in one or more biological samples from one or more transgenic plants, the surrogate peptide comprising a label and an amino acid sequence selected from the group consisting of GSAQGIEGSIR (SEQ ID NO:1), IVAQLGQGVYR (SEQ ID NO:2), TLSSTLYR (SEQ ID NO:3), DVSVFGQR (SEQ ID NO:4), TYPIR (SEQ ID NO:5), TVSQLTR (SEQ ID NO:6), WYNTGLER (SEQ ID NO:7), EWEADPTNPALR (SEQ ID NO:8), VWGPDSR (SEQ ID NO:9), APMFSWIHR (SEQ ID NO:10), WGFDAATINSR (SEQ ID NO:11), NQAISR (SEQ ID NO:12), IEEFAR (SEQ ID NO:13), SGFSNSSVSIIR (SEQ ID NO:14), LSHVSMFR (SEQ ID NO:15), EIYTNPVLENFDGSFR (SEQ ID NO:16), LEGLSNLYQIYAESFR (SEQ ID NO:17), YNQFR (SEQ ID NO:18), YNDLTR (SEQ ID NO:19), SPHLMDILNSITIYTDAHR (SEQ ID NO:20), SAEFNNIIPSSQITQIPLTK (SEQ ID NO:21), QGFSHR (SEQ ID NO:22), MDNNPNINECIPYNCLSNPEVEVLGGER (SEQ ID NO:23), ELTLTVLDIVSLFPNYDSR (SEQ ID NO:24), RPFNIGINNQQLSVLDGTEFAYGTSSNLPSAVYR (SEQ ID NO:25), SGTVDSLDEIPPQNNNVPPR (SEQ ID NO:26), TDVTDYHIDQV (SEQ ID NO:27), AVNELFTSSNQIGLK (SEQ ID NO:28), ITQLPLTK (SEQ ID NO:29), GLDSSTTK (SEQ ID NO:30), QCAGIRPYDGR (SEQ ID NO:31), IEFVPAEVTFEAEYDLER (SEQ ID NO:32), ITQLPLVK (SEQ ID NO:33), MTADNNTEALDSSTTK (SEQ ID NO:34), VYIDK (SEQ ID NO:35), DGGISQFIGDK (SEQ ID NO:36), LITLTCK (SEQ ID NO:37), ELLLATDLSNK (SEQ ID NO:38), FEELTFATETSSK (SEQ ID NO:39), EVLFEK (SEQ ID NO:40), TASELITK (SEQ ID NO:41), DVSEMFTTK (SEQ ID NO:42), LLGLADIDYTSIMNEHLNK (SEQ ID NO:43), IDFTK (SEQ ID NO:44), TDTGGDLTLDEILK (SEQ ID NO:45), DIMNMIFK (SEQ ID NO:46), ALYVHK (SEQ ID NO:47), VNILPTLSNTFSNPNYAK (SEQ ID NO:48), ITSMLSDVIK (SEQ ID NO:49), QNLQLDSFSTYR (SEQ ID NO:50), DSLSEVIYGDMDK (SEQ ID NO:51), MIVEAKPGHALIGFEISNDSITVLK (SEQ ID NO:52), VYFSVSGDANVR (SEQ ID NO:53), NQQLLNDISGK (SEQ ID NO:54), VESSEAEYR (SEQ ID NO:55), YMSGAK (SEQ ID NO:56), DGSPADILDELTELTELAK (SEQ ID NO:57), VYEAK (SEQ ID NO:58), LDAINTMLR (SEQ ID NO:59), GKPSIHLK (SEQ ID NO:60), DENTGYIHYEDTNNNLEDYQTINK (SEQ ID NO:61), DNFYIELSQGNNLYGGPIVHFYDVSIK (SEQ ID NO:62), LLCPDQSEQIYYTNNIVFPNEYVITK (SEQ ID NO:63), SQNGDEAWGDNFIILEISPSEK (SEQ ID NO:64), NAYVDHTGGVNGTK (SEQ ID NO:65), LDGVNGSLNDLIAQGNLNTELSK (SEQ ID NO:66), IANEQNQVLNDVNNK (SEQ ID NO:67), YEVTANFYDSSTGEIDLNK (SEQ ID NO:68), QNYALSLQIEYLSK

(SEQ ID NO:69), QLQEISDK (SEQ ID NO:70), LLSPELINTNNWTSTGSTNISGNTLTLYQGGR (SEQ ID NO:71), YVNEK (SEQ ID NO:72), QNYQVDK (SEQ ID NO:73), MAGAEEIVLQPIK (SEQ ID NO:74), FPVEDAK (SEQ ID NO:75), EISGTVK (SEQ ID NO:76), ILLLAALSEGTTVVDNLLNSEDVHYMLGALR (SEQ ID NO:77), DFELPAPPRPVRPVTQI (SEQ ID NO:78), LGLGSTLYTHLLK (SEQ ID NO:79), MSPER (SEQ ID NO:80), HGGWHDVGFWQR (SEQ ID NO:81), NAYDWTVESTVYVSHR (SEQ ID NO:82), TEPQTPQEWIDDLER (SEQ ID NO:83), AAGYK (SEQ ID NO:84), YPWLVAEVEGVVAGIAYAGPWK (SEQ ID NO:85), RPVEIRPATAADMAAVCDIVNHYIETSTVNFR (SEQ ID NO:86), ENAAGIPMDAAER (SEQ ID NO:87), ALAILK (SEQ ID NO:88), SALDSQQGEPWQTIR (SEQ ID NO:89), GSQQLQLKPGESAFIAANESPVTVK (SEQ ID NO:90), FEAKPANQLLTQPVK (SEQ ID NO:91), STLLGEAVAK (SEQ ID NO:92), LINSVQNYAWGSK (SEQ ID NO:93), HNSEIGFAK (SEQ ID NO:94), VLCAAQPLSIQVHPNK (SEQ ID NO:95), TALTELYGMENPSSQPMAELWMGAHPK (SEQ ID NO:96), LSELFASLLNMQGEEK (SEQ ID NO:97) and QGAELDFPIPVDDFAFSLHDLSDK (SEQ ID NO:98). In other embodiments, the surrogate peptide is labeled by incorporation of a stable isotope labeled (SIL) amino acid. In other embodiments, the SIL peptides are labeled by incorporation of [¹³C₆¹⁵N₂] lysine, [¹³C₆¹⁵N₂] isoleucine, [¹³C₆¹⁵N₂] valine or [¹³C₆¹⁵N₂] arginine.

In some embodiments, the labeled surrogate peptide selectively detects or quantitates a Cry1Ab protein in the mixture of transgenic and non-transgenic proteins and comprises an amino acid sequence selected from the group consisting of GSAQGIEGSIR (SEQ ID NO:1), IVAQLGQGVYR (SEQ ID NO:2), TLSSTLYR (SEQ ID NO:3), DVSVFGQR (SEQ ID NO:4), TYPIR (SEQ ID NO:5), TVSQLTR (SEQ ID NO:6), WYNTGLER (SEQ ID NO:7), EWEADPTNPALR (SEQ ID NO:8), VWGPDSR (SEQ ID NO:9), APMFSWIHR (SEQ ID NO:10), WGFDAATINSR (SEQ ID NO:11), NQAISR (SEQ ID NO:12), IEEFAR (SEQ ID NO:13), SGFSNSSVSIIR (SEQ ID NO:14), LSHVSMFR (SEQ ID NO:15), EIYTNPVLENFDGSFR (SEQ ID NO:16), LEGLSNLYQIYAESFR (SEQ ID NO:17), YNQFR (SEQ ID NO:18), YNDLTR (SEQ ID NO:19), SPHLMDILNSITIYTDAHR (SEQ ID NO:20), SAEFNNIIPSSQITQIPLTK (SEQ ID NO:21), QGFSHR (SEQ ID NO:22), MDNNPNINECIPYNCLSNPEVEVLGGER (SEQ ID NO:23), ELTLTVLDIVSLFPNYDSR (SEQ ID NO:24), RPFNIGINNQQLSVLDGTEFAYGTSSNLPSAVYR (SEQ ID NO:25) and SGTVDSLDEIPPQNNNVPPR (SEQ ID NO:26).

In other embodiments, the Cry1Ab-specific labeled surrogate peptide of the invention produces a transition ion having an amino acid sequence selected from the group consisting of GIEGSIR (SEQ ID NO:99), EGSIR (SEQ ID NO:100), AQLGQGVYR (SEQ ID NO:101), GQGVYR (SEQ ID NO:102); SSTLYR (SEQ ID NO:103), STLYR (SEQ ID NO:104), SVFGQR (SEQ ID NO:105), FGQR (SEQ ID NO:106), PIR, TY, SQLTR (SEQ ID NO:107), QLTR (SEQ ID NO:108), NTGLER (SEQ ID NO:109), YNTGLER (SEQ ID NO:110), PTNPALR (SEQ ID NO:111), DPTNPALR (SEQ ID NO:112), GPDSR (SEQ ID NO:113), VW, HR, SWIHR (SEQ ID NO:114), ATINSR (SEQ ID NO:115), DAATINSR (SEQ ID NO:116), AISR (SEQ ID NO:117), ISR, EFAR (SEQ ID NO:118), EEFAR (SEQ ID NO:119), SNSSVSIIR (SEQ ID NO:120), SSVSIIR (SEQ ID NO:121), SMFR (SEQ ID NO:122), VSMFR (SEQ ID NO:123), ENFDGSFR (SEQ ID NO:124), GSFR (SEQ ID NO:125), YAESFR (SEQ ID NO:126), LEG, NQFR (SEQ ID NO:127), QFR, DLTR (SEQ ID NO:128), NDLTR (SEQ ID NO:129), TIYTDAHR (SEQ ID NO:130), YTDAHR (SEQ ID NO:131), PLTK (SEQ ID NO:132), SAEFNNII (SEQ ID NO:133), FSHR (SEQ ID NO:134), GFSHR (SEQ ID NO:135), EVLGGER (SEQ ID NO:136), GGER (SEQ ID NO:137), FPNYDSR (SEQ ID NO:138), PNYDSR (SEQ ID NO:139), PSAVYR (SEQ ID NO:140), YR, PPR, and SGTVDSLDE (SEQ ID NO:141).

In still other embodiments, a Cry1Ab-specific labeled surrogate peptide of the invention comprises the amino acid sequence SAEFNNIIPSSQITQIPLTK (SEQ ID NO:21) and produces a transition ion consisting of the amino acid sequence PLTK (SEQ ID NO:132) or SAEFNNII (SEQ ID NO:133).

In some embodiments, a labeled surrogate peptide of the invention selectively detects or quantitates an eCry3.1Ab protein and comprises an amino acid sequence selected from the group consisting of TDVTDYHIDQV (SEQ ID NO:27), AVNELFTSSNQIGLK (SEQ ID NO:28), ITQLPLTK (SEQ ID NO:29), GLDSSTTK (SEQ ID NO:30), QCAGIRPYDGR (SEQ ID NO:31) and IEFVPAEVTFEAEYDLER (SEQ ID NO:32).

In other embodiments, the eCry3.1Ab-specific labeled surrogate peptide produces a transition ion having an amino acid sequence selected from the group consisting of TDYHIDQV (SEQ ID NO:142), DYHIDQV (SEQ ID NO:143), TSSNQIGLK (SEQ ID NO:144), SSNQIGLK (SEQ ID NO:145), QLPLTK (SEQ ID NO:146), TQLPLTK (SEQ ID NO:147), DSSTTK (SEQ ID NO:148), SSTTK (SEQ ID NO:149), PYDGR (SEQ ID NO:150), DGR, IEF, and LER.

In still other embodiments, an eCry3.1Ab-specific labeled surrogate peptide of the invention comprises the amino acid sequence TDVTDYHIDQV (SEQ ID NO:27) and produces a transition ion consisting of the amino acid sequence TDYHIDQV (SEQ ID NO:142) or DYHIDQV (SEQ ID NO:143).

In some embodiments, the labeled surrogate peptide selectively detects or quantitates a mCry3A protein and comprises an amino acid sequence selected from the group consisting of ITQLPLVK (SEQ ID NO:33), MTADNNTEALDSSTTK (SEQ ID NO:34), VYIDK (SEQ ID NO:35) and LQSGASVVAGPR (SEQ ID NO:252).

In other embodiments, the mCry3A-specific surrogate peptide produces a transition ion having an amino acid sequence selected from the group consisting of QLPLVK (SEQ ID NO:151), TQLPLVK (SEQ ID NO:152), ALDSSTTK (SEQ ID NO:153), EALDSSTTK (SEQ ID NO:154), YIDK (SEQ ID NO:155) and IDK.

In still other embodiments, a mCry3A-specific labeled surrogate peptide of the invention comprises the amino acid sequence LQSGASVVAGPR (SEQ ID NO:252) and produces a transition ion consisting of the amino acid sequence SGASVVAGPR (SEQ ID NO:253) and SVVAGPR (SEQ ID NO:254).

In some embodiments, the labeled surrogate peptide of the invention selectively detects or quantitates a Vip3A protein and comprises an amino acid sequence selected from the group consisting of DGGISQFIGDK (SEQ ID NO:36), LITLTCK (SEQ ID NO:37), ELLLATDLSNK (SEQ ID NO:38), FEELTFATETSSK (SEQ ID NO:39), EVLFEK (SEQ ID NO:40), TASELITK (SEQ ID NO:41), DVSEMFTTK (SEQ ID NO:42), LLGLADIDYTSIMNEHLNK (SEQ ID NO:43), IDFTK (SEQ ID NO:44), TDTGGDLTLDEILK (SEQ ID NO:45), DIMNMIFK (SEQ ID NO:46), ALYVHK (SEQ ID NO:47), VNILPTLSNTFSNPNYAK (SEQ ID NO:48), ITSMLSDVIK (SEQ ID NO:49), QNLQLDSFSTYR (SEQ ID NO:50), DSLSEVIYGDMDK (SEQ ID NO:51), MIVEAKPGHALIGFEISNDSITVLK (SEQ ID NO:52), VYFSVSGDANVR (SEQ ID NO:53), NQQLLNDISGK (SEQ ID NO:54), VESSEAEYR (SEQ ID NO:55), YMSGAK (SEQ ID NO:56), DGSPADILDELTELTELAK (SEQ ID NO:57), VYEAK (SEQ ID NO:58), LDAINTMLR (SEQ ID NO:59), GKPSIHLK (SEQ ID NO:60), DENTGYIHYEDTNNNLEDYQTINK (SEQ ID NO:61), DNFYIELSQGNNLYGGPIVHFYDVSIK (SEQ ID NO:62), LLCPDQSEQIYYTNNIVFPNEYVITK (SEQ ID NO:63), SQNGDEAWGDNFIILEISPSEK (SEQ ID NO:64), NAYVDHTGGVNGTK (SEQ ID NO:65), LDGVNGSLNDLIAQGNLNTELSK (SEQ ID NO:66), IANEQNQVLNDVNNK (SEQ ID NO:67), YEVTANFYDSSTGEIDLNK (SEQ ID NO:68), QNYALSLQIEYLSK (SEQ ID NO:69), QLQEISDK (SEQ ID NO:70), LLSPELINTNNWTSTGSTNISGNTLTLYQGGR (SEQ ID NO:71), YVNEK (SEQ ID NO:72), QNYQVDK (SEQ ID NO:73) and FTTGTDLK (SEQ ID NO:255).

In other embodiments, the Vip3A-specific labeled surrogate peptide produces a transition ion having an amino acid sequence selected from the group consisting of SQFIGDK (SEQ ID NO:156), GDK, TLTCK (SEQ ID NO:157), TCK, ATDLSNK (SEQ ID NO:158), LATDLSNK (SEQ ID NO:159), TFATETSSK (SEQ ID NO:160), FATETSSK (SEQ ID NO:161), FEK, LFEK (SEQ ID NO:162), SELITK (SEQ ID NO:163), ASELITK (SEQ ID NO:164), SEMFTTK (SEQ ID NO:165), DVS, IMNEHLNK (SEQ ID NO:166), MNEHLNK (SEQ ID NO:167), DFTK (SEQ ID NO:168), FTK, TLDEILK (SEQ ID NO:169), LTLDEILK (SEQ ID NO:170), MNMIFK (SEQ ID NO:171), NMIFK (SEQ ID NO:172), YVHK (SEQ ID NO:173), HK, VNI, VNIL (SEQ ID NO:174), SMLSDVIK (SEQ ID NO:175), TSMLSDVIK (SEQ ID NO:176), DSFSTYR (SEQ ID NO:177), LDSFSTYR (SEQ ID NO:178), IYGDMDK (SEQ ID NO:179), VIYGDMDK (SEQ ID NO:180), SNDSITVLK (SEQ ID NO:181), MIV, SGDANVR (SEQ ID NO:182), SVSGDANVR (SEQ ID NO:183), LLNDISGK (SEQ ID NO:184), LNDISGK (SEQ ID NO:185), SSEAEYR (SEQ ID NO:186), ESSEAEYR (SEQ ID NO:187), SGAK (SEQ ID NO:188), MSGAK (SEQ ID NO:189), TELTELAK (SEQ ID NO:190), DGSPADI (SEQ ID NO:191), YEAK (SEQ ID NO:192), EAK, NTMLR (SEQ ID NO:193), AINTMLR (SEQ ID NO:194), PSIHLK (SEQ ID NO:195), HLK, DYQTINK (SEQ ID NO:196), NK, DNF, DNFY (SEQ ID NO:197), PNEYVITK (SEQ ID NO:198), LLC, SPSEK (SEQ ID NO:199), LEISPSEK (SEQ ID NO:200), NAY, DHTGGVNGTK (SEQ ID NO:201), GNLNTELSK (SEQ ID NO:202), NTELSK (SEQ ID NO:203), LNDVNNK (SEQ ID NO:204), NDVNNK (SEQ ID NO:205), YE, DLNK (SEQ ID NO:206), QIEYLSK (SEQ ID NO:207), LQIEYLSK (SEQ ID NO:208), SDK, QEISDK (SEQ ID NO:209), YQGGR (SEQ ID NO:210), TLYQGGR (SEQ ID NO:211), NEK, VNEK (SEQ ID NO:212), DK, and VDK.

In still other embodiments, a Vip3A-specific labeled surrogate peptide of the invention comprises the amino acid sequence FTTGTDLK (SEQ ID NO:255) and produces a transition ion consisting of the amino acid sequence TGTDLK (SEQ ID NO:256) and LK.

In some embodiments, the labeled surrogate peptide of the invention selectively detects or quantitates a dmEPSPS protein and comprises an amino acid sequence selected from the group consisting of MAGAEEIVLQPIK (SEQ ID NO:74), FPVEDAK (SEQ ID NO:75), EISGTVK (SEQ ID NO:76), ILLLAALSEGTTVVDNLLNSEDVHYMLGALR (SEQ ID NO:77) and SLTAAVTAAGGNATYVLDGVPR (SEQ ID NO:257).

In other embodiments, the EPSPS-specific labeled surrogate peptide produces a transition ion having an amino acid sequence selected from the group consisting of PIK, EIVLQPIK (SEQ ID NO:213), PVEDAK (SEQ ID NO:214), VEDAK (SEQ ID NO:215), SGTVK (SEQ ID NO:216), GTVK (SEQ ID NO:217), ILLLAA (SEQ ID NO:218), and HYMLGALR (SEQ ID NO:219).

In still other embodiments, a dmEPSPS-specific labeled surrogate peptide of the invention comprises the amino acid sequence SLTAAVTAAGGNATYVLDGVPR (SEQ ID NO:257) and produces a transition ion consisting of the amino acid sequence PR and GVPR (SEQ ID NO:258).

In some embodiments, the labeled surrogate peptide of the invention selectively detects or quantitates a PAT protein and comprises an amino acid sequence selected from the group consisting of DFELPAPPRPVRPVTQI (SEQ ID NO:78), LGLGSTLYTHLLK (SEQ ID NO:79), MSPER (SEQ ID NO:80), HGGWHDVGFWQR (SEQ ID NO:81), NAYDWTVESTVYVSHR (SEQ ID NO:82), TEPQTPQEWIDDLER (SEQ ID NO:83), AAGYK (SEQ ID NO:84), YPWLVAEVEGVVAGIAYAGPWK (SEQ ID NO:85) and RPVEIRPATAADMAAVCDIVNHYIETSTVNFR (SEQ ID NO:86).

In other embodiment, the PAT-specific labeled surrogate peptide produces a transition ion having an amino acid sequence selected from the group consisting of DFE, DF, YTHLLK (SEQ ID NO:220), THLLK (SEQ ID NO:221), PER, SPER (SEQ ID NO:222), GFWQR (SEQ ID NO:223), VGFWQR (SEQ ID NO:224), STVYVSHR (SEQ ID NO:225), SHR, TEPQT (SEQ ID NO:226), DLER (SEQ ID NO:227), GYK, AGYK (SEQ ID NO:228), GPWK (SEQ ID NO:229) GIAYAGPWK (SEQ ID NO:230), TSTVNFR (SEQ ID NO:231), and NFR.

In still other embodiments, the PAT-specific labeled surrogate peptide comprises the amino acid sequence LGLGSTLYTHLLK (SEQ ID NO:79) and produces a transition ion consisting of the amino acid sequence YTHLLK (SEQ ID NO:220) or THLLK (SEQ ID NO:221).

In some embodiments, a labeled surrogate peptide of the invention selectively detects or quantitates a PMI protein and comprises an amino acid sequence selected from the group consisting of ENAAGIPMDAAER (SEQ ID NO:87), ALAILK (SEQ ID NO:88), SALDSQQGEPWQTIR (SEQ ID NO:89), GSQQLQLKPGESAFIAANESPVTVK (SEQ ID NO:90), FEAKPANQLLTQPVK (SEQ ID NO:91), STLLGEAVAK (SEQ ID NO:92), LINSVQNYAWGSK (SEQ ID NO:93), HNSEIGFAK (SEQ ID NO:94), VLCAAQPLSIQVHPNK (SEQ ID NO:95), TALTELYGMENPSSQPMAELWMGAHPK (SEQ ID NO:96), LSELFASLLNMQGEEK (SEQ ID NO:97) and QGAELDFPIPVDDFAFSLHDLSDK (SEQ ID NO:98).

In other embodiments, the PMI-specific labeled surrogate peptide produces a transition ion having an amino acid sequence selected from the group consisting of PMDAAER (SEQ ID NO:232), GIPMDAAER (SEQ ID NO:233), AILK (SEQ ID NO:234), LK, PWQTIR (SEQ ID NO:235), GEPWQTIR (SEQ ID NO:236), ANESPVTVK (SEQ ID NO:237), PVTVK (SEQ ID NO:238), LTQPVK (SEQ ID NO:239), PVK, GEAVAK (SEQ ID NO:240), LGEAVAK (SEQ ID NO:241), QNYAWGSK (SEQ ID NO:242), NYAWGSK (SEQ ID NO:243), NSEIGFAK (SEQ ID NO:244), HN, VLCAAQ (SEQ ID NO:245), PNK, WMGAHPK (SEQ ID NO:246), TALTE (SEQ ID NO:247), NMQGEEK (SEQ ID NO:248) LNMQGEEK (SEQ ID NO:249), SLHDLSDK (SEQ ID NO:250), and HDLSDK (SEQ ID NO:251).

In still other embodiments, the PMI-specific surrogate peptide comprises the amino acid sequence SALDSQQGEPWQTIR (SEQ ID NO:89) and produces a transition ion consisting of the amino acid sequence PWQTIR (SEQ ID NO:235) or GEPWQTIR (SEQ ID NO:236).

According to some embodiments, a Cry1Ab-specific labeled surrogate peptide of the invention detects and/or quantitates a Cry1Ab protein comprising the amino acid sequence of SEQ ID NO:259. In other embodiments, the Cry1Ab protein is from the transgenic corn event Bt11.

In some embodiments, an eCry3.1Ab-specific labeled surrogate peptide of the invention detects and/or quantitates an eCry3.1Ab protein comprising the amino acid sequence of SEQ ID NO:260. In other embodiments, the eCry3.1Ab protein is from transgenic corn event 5307.

According to some embodiments, a mCry3A-specific labeled surrogate peptide of the invention detects and/or quantitates a mCry3A protein comprising the amino acid sequence of SEQ ID NO:261. In other embodiments, the mCry3A protein is from the transgenic corn event MIR604.

According to some embodiments, a Vip3-specific labeled surrogate peptide of the invention detects and/or quantitates a Vip3Aa protein comprising the amino acid sequence of SEQ ID NO:262. In other embodiments, the Vip3Aa protein is from the transgenic corn event MIR162.

According to some embodiments, a dmEPSPS-specific labeled surrogate peptide of the invention detects and/or quantitates a dmEPSPS protein comprising the amino acid sequence of SEQ ID NO:263. In other embodiments, the dmEPSPS protein is from the transgenic corn event GA21.

According to some embodiments, a PAT-specific labeled surrogate peptide of the invention detects and/or quantitates a PAT protein comprising the amino acid sequence of SEQ ID NO:264. In other embodiments, the PAT protein is from the transgenic corn event Bt11, 59122, TC1507, DP4114 or T25.

According to some embodiments, a PMI-specific labeled surrogate peptide of the invention detects and/or quantitates a PMI protein comprising the amino acid sequence of SEQ ID NO:265 or SEQ ID NO:266. In other embodiments, the PMI protein is from the transgenic corn event MIR162, MIR604, 5307 or 3272.

In some embodiments, the labeled surrogate peptide of the invention specifically detects or quantitates a Cry1Ab protein, an eCry3.1Ab protein, an mCry3A protein, a Vip3 protein, a dmEPSPS protein, a PAT protein or a PMI protein in a mixture of transgenic proteins that comprises at least two transgenic proteins selected from the group consisting of a Cry1Ab protein, an eCry3.1Ab protein, a mCry3A protein, a Vip3A protein, a dmEPSPS protein, a PAT protein and a PMI protein. In other embodiments, the mixture of transgenic proteins comprises a Cry1Ab protein, an eCry3.1Ab protein, a mCry3A protein, a Vip3A protein, a dmEPSPS protein, a PAT protein and a PMI protein. In still other embodiments, the mixture of transgenic proteins further comprises at least one transgenic protein selected from the group consisting of a Cry1A.105 protein (SEQ ID NO:267), a Cry1F protein (SEQ ID NO:268), a Cry34 protein (SEQ ID NO:269) and a Cry35 protein (SEQ ID NO:270).

In some embodiments, the labeled surrogate peptide of the invention specifically detects or quantitates a Cry1Ab protein, an eCry3.1Ab protein, a mCry3A protein, a Vip3 protein, a dmEPSPS protein, a PAT protein or a PMI protein in a mixture of transgenic proteins in a biological sample from a transgenic plant, wherein the transgenic plant is a corn plant, soybean plant, cotton plant, rice plant, wheat plant or canola plant. In other embodiments, the transgenic plant is a corn plant that comprises a transgenic event selected from the group consisting of event Bt11, event 5307, event MIR604, event MIR162, event 3272 and event GA21. In still other embodiments, the transgenic corn plant further comprises event MON89034, event DP4114, event TC1507, event 59122 or event T25.

In some embodiments, the labeled surrogate peptide of the invention specifically detects or quantitates a Cry1Ab protein, an eCry3.1Ab protein, a mCry3A protein, a Vip3 protein, a dmEPSPS protein, a PAT protein or a PMI protein in a biological sample from leaf tissue, seed, grain, pollen, or root tissue from a transgenic plant. In other embodiments, the leaf tissue, seed, grain, pollen or root tissue is from a transgenic corn plant comprising one or more of the transgenic corn events Bt11, 5307, MIR604, MIR162, GA21, 3272, 59122, DP4114, TC1507 and T25.

There are many references in the art that have suggested many different methods of predicting which surrogate peptides are the best for any given target protein and many references have suggested shortcuts to quantifying target proteins using mass spectrometry, e.g. Mead et al. 2009. Mol. Cell. Proteomics 8:696-705 and U.S. Pat. No. 8,227,252. However, reliance on such prediction methods and shortcuts can lead to confounding results, because unpredictable factors can interfere with the mass spectrometry based assay thus causing a loss of sensitivity and inaccurate quantification. At least one primary factor lies in the biological matrix itself. For example, it is very unpredictable and difficult to identify a single transition ion from a surrogate peptide that will work equally well with biological samples from leaves, roots, pollen and seeds from transgenic plants. Differences in chemical composition, pH, or ionic strength of the matrix can influence proteolysis, peptide stability, aggregation, or ionization in an MS instrument. Therefore, identifying and empirically testing surrogate peptides and specific surrogate peptide/transition ion combination across all relevant matrices, particularly those for transgenic plants is imperative to overcome the unpredictable nature of such assays. The present invention employs a two-step approach in developing mass spectrometry assays for specifically detecting and/or quantitating target transgenic proteins, including 1) testing and selecting surrogate peptides from a pool of peptides derived from a proteolytically cleaved target protein and testing combinations of SIL surrogate peptides and transition ion peptides and selecting the combination that specifically detects and quantitates the target protein across all biological matrices, for example biological samples from leaves, roots, pollen or seeds of transgenic plants; and 2) empirically determining appropriate methods of sample preparation and mass spectrometer conditions that work for all surrogate peptides and surrogate peptide/transition ion combinations in all biological matrices, including leaves, roots, pollen and seeds of transgenic plants, particularly transgenic corn plants.

Therefore, in some embodiments, the present invention encompasses a method of simultaneously detecting and/or quantitating one or more target transgenic proteins in a complex biological sample from a transgenic plant comprising a mixture of the target transgenic proteins and non-transgenic proteins, where the method comprises the following steps: a) obtaining a biological sample from a transgenic plant; b) extracting proteins from the biological sample, resulting in an extract comprising a mixture of proteins; c) reducing the amount of non-transgenic insoluble proteins in the extract of step b, resulting in an extract of concentrated soluble proteins; d) digesting the soluble proteins in the extract of step c, resulting in an extract comprising peptide fragments, wherein the peptide fragments include at least one non-labeled surrogate peptide specific for each target transgenic protein; e) concentrating the peptide fragments in the extract of step d; f) adding one or more labeled surrogate peptides of the invention, wherein each labeled surrogate peptide has the same amino acid sequence as each non-labeled surrogate peptide derived from the target transgenic proteins, and wherein the number of labeled surrogate peptides that are added is equal to the number of target transgenic proteins in the mixture; g) concentrating the non-labeled surrogate peptides and the labeled surrogate peptides by reducing the amount of non-surrogate peptides in the mixture; h) resolving the peptide fragment mixture from step g via liquid chromatography; i) analyzing the peptide fragment mixture resulting from step h via mass spectrometry, wherein detection of a transition ion fragment of a non-labeled surrogate peptide is indicative of the presence of a target transgenic protein from which the surrogate peptide is derived; and optionally, j) calculating an amount of a target transgenic protein in the biological sample by comparing mass spectrometry signals generated from the transition ion fragment of step i with mass spectrometry signals generated by a transition ion of a labeled surrogate peptide.

In some embodiments, the target transgenic protein that is detected and/or quantitated by the above-described method is a Cry1Ab protein, an eCry3.1Ab protein, a mCry3A protein, a Vip3 protein, a double mutant 5-enolpyruvylshikimate-3-phosphate synthase (dmEPSPS) protein, a phosphinothricin acetyltransferase (PAT) protein or a phosphomannose isomerase (PMI) protein.

In other embodiments encompassed by a method of the invention, the target transgenic protein is Cry1Ab and the labeled surrogate peptide comprises the amino acid sequence SAEFNNIIPSSQITQIPLTK (SEQ ID NO:21) and produces a transition ion consisting of the amino acid sequence PLTK (SEQ ID NO:132) or SAEFNNII (SEQ ID NO:133). In still other embodiments, the Cry1Ab target protein is quantitated by comparing mass spectrometry signals generated from a non-labeled and labeled transition ion consisting of the amino acid sequence PLTK (SEQ ID NO:132).

In other embodiments encompassed by a method of the invention, the target transgenic protein is eCry3.1Ab and the labeled surrogate peptide comprises the amino acid sequence TDVTDYHIDQV (SEQ ID NO:27) and produces a transition ion consisting of the amino acid sequence TDYHIDQV (SEQ ID NO:142) or DYHIDQV (SEQ ID NO:143). In still other embodiments, the eCry3.1Ab target protein is quantitated by comparing mass spectrometry signals generated from a non-labeled and labeled transition ion consisting of the amino acid sequence TDYHIDQV (SEQ ID NO:142).

In other embodiments encompassed by a method of the invention, the target transgenic protein is mCry3A and the labeled surrogate peptide comprises the amino acid sequence LQSGASVVAGPR (SEQ ID NO:252) and produces a transition ion consisting of the amino acid sequence SGASVVAGPR (SEQ ID NO:253) or SVVAGPR (SEQ ID NO:254). In still other embodiments, the mCry3A target protein is quantitated by comparing mass spectrometry signals generated from a non-labeled and labeled transition ion consisting of the amino acid sequence SGASVVAGPR (SEQ ID NO:253).

In other embodiments encompassed by a method of the invention, the target transgenic protein is Vip3A and the labeled surrogate peptide comprises the amino acid sequence FTTGTDLK (SEQ ID NO:255) and produces a transition ion consisting of the amino acid sequence TGTDLK (SEQ ID NO:256) or LK. In still other embodiments, the Vip3A target protein is quantitated by comparing mass spectrometry signals generated from a non-labeled and labeled transition ion consisting of the amino acid sequence TGTDLK (SEQ ID NO:256).

In other embodiments encompassed by a method of the invention, the target transgenic protein is dmEPSPS and the labeled surrogate peptide comprises the amino acid sequence SLTAAVTAAGGNATYVLDGVPR (SEQ ID NO:257) and produces a transition ion consisting of the amino acid sequence PR or GVPR (SEQ ID NO:258). In still other embodiments, the eCry3.1Ab target protein is quantitated by comparing mass spectrometry signals generated from a non-labeled and labeled transition ion consisting of the amino acid sequence PR.

In other embodiments encompassed by a method of the invention, the target transgenic protein is PAT and the labeled surrogate peptide comprises the amino acid sequence LGLGSTLYTHLLK (SEQ ID NO:79) and produces a transition ion consisting of the amino acid sequence YTHLLK (SEQ ID NO:220) or THLLK (SEQ ID NO:221). In still other embodiments, the PAT target protein is quantitated by comparing mass spectrometry signals generated from a non-labeled and labeled transition ion consisting of the amino acid sequence YTHLLK (SEQ ID NO:220).

In other embodiments encompassed by a method of the invention, the target transgenic protein is PMI and the labeled surrogate peptide comprises the amino acid sequence SALDSQQGEPWQTIR (SEQ ID NO:89) and produces a transition ion consisting of the amino acid sequence PWQTIR (SEQ ID NO:235) or GEPWQTIR (SEQ ID NO:236). In still other embodiments, the PMI target protein is quantitated by comparing mass spectrometry signals generated from a non-labeled and labeled transition ion consisting of the amino acid sequence PWQTIR (SEQ ID NO:235).

In other embodiments, the invention encompasses a system for high-throughput detection or quantitation of transgenic target proteins. Such system comprises a cassette of pre-designed labelled surrogate peptides that are specific for the transgenic target proteins; and one or more mass spectrometers. In one aspect of this embodiment, the cassette comprises a labelled surrogate peptide that is specific for a target protein selected from the group consisting of Cry1Ab, eCry3.1Ab, mCry3A, Vip3, dmEPSPS, PAT and PMI. In other aspects of this embodiment, the labelled surrogate peptide comprises any one of SEQ ID NOs:1-98. In other aspects of this embodiment the labelled surrogate peptide produces one or more transition ions comprising a peptide sequence selected from the group consisting of at least one of SEQ ID NOs:99-251, SEQ ID NOs:254, 255, 256, the peptides PIR, TY, VW, HR, ISR, LEG, QFR, YR, PPR, DGR, IEF, LER, IDK, GDK, TCK, FEK, DVS, FTK, HK, VNI, MIV, EAK, HLK, NK, DNF, LLC, NAY, YE, SDK, NEK, DK, VDK, PIK, DFE, DF, PER, SHR, GYK, NFR, LK, PVK, HN, PNK and PR.

The following specific examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Examples

### Example 1—Surrogate Peptide Selection

MRM-based assays rely on selecting a predetermined set of peptides and depend upon specific fragmentation/transition ions for each selected surrogate peptide. Several criteria are required to select suitable surrogate or signature peptides. First, the proteins that constitute the targeted protein cassette have to be selected. Second, for each target protein, those peptides that present good mass spectrometry responses and uniquely identify the target protein, or a specific modification (i.e. post translational modification) thereof, have to be identified. Third, for each mass spectrometry suitable peptide, those transition ions that provide optimal signal intensity and uniquely differentiate the surrogate peptide from other peptide species present in the sample have to be identified. These criteria are essential to perform a MRM-based assay.

Surrogate peptides from seven transgenic proteins, Cry1Ab, eCry3.1Ab, mCry3A, Vip3A, dmEPSPS, PAT and PMI, were identified and selected for MRM-based assays. The MRM assay was developed using microbe-produced proteins that were digested with trypsin. The microbe-produced proteins were individually reconstituted with water. Trifluoroethanol (TFE) was then added to an aliquot of each protein, followed by addition of 100 mM ammonium bicarbonate and trypsin (1:10 (w:w) enzyme:protein ratio). The samples were digested overnight at about 37° C. followed by addition of about 0.05 M tris(2-carboxyethyl)phosphine (TCEP). Each protein was aliquoted to create a pool with a final concentration of about 200 pmol/μL. This peptide mix was used to develop the MRM assay on a QTRAP 6500 mass spectrometer (AB Sciex LLC, Framingham, Mass. USA). The optimal two transitions (combination of peptide surrogate and fragment ion mass-to-charge (m/z) ratio that are monitored by the mass spectrometer) per peptide were determined using selected reaction monitoring MS/MS. The MS/MS method was developed by calculating, for each peptide, the signature mass of the doubly and triply charged peptide ions and the first and second y fragment ion with an m/z greater than [m/z (surrogate)+20 Da]. If these calculated transitions were observed during the MRM scan, the instrument switched automatically to MS/MS mode and acquired a full MS/MS spectrum of the surrogate peptide ion. The two most intense fragment ions (b or y fragment ions only) in the MS/MS spectrum and its elution time were determined for each acquired peptide. The collision energy (CE) was then optimized for each of the chosen transitions. The developed MRM assay was utilized for the analysis of the calibration curve samples.

The MRM assay targeted 193 proteotypic peptides from the seven transgenic proteins. Of these, 111 peptides were unique to the seven proteins and did not overlap with known maize proteins. Table 1 lists the characteristics of surrogate peptides and transition ions for each target protein including amino acid sequence (including sequence listing identifiers for peptides comprising at least four amino acids), monoisotopic mass, signature charge state, signature m/z, and the product transition m/z. Unique surrogate peptides were identified for all seven proteins; Cry1Ab (26), eCry3.1Ab (6), mCry3A (4), Vip3Aa20 (39), dmEPSPS (5), PAT (9) and PMI (12). These surrogate peptides from Cry1Ab, eCry3.1Ab, mCry3A, Vip3A, dmEPSPS, PAT and PMI were identified as useful in the determination of absolute or relative amounts for Cry1Ab, eCry3.1Ab, mCry3A, Vip3A, dmEPSPS, PAT and PMI transgenic proteins. Each of these peptides or combinations of peptides listed in Table 1 were detected by mass spectrometry in lysates and are potential candidates for use in MRM-based assays for the quantitation of Cry1Ab, eCry3.1Ab, mCry3A, Vip3A, dmEPSPS, PAT and PMI.

TABLE 1
Characteristics of surrogate peptides and transition ions.

| | | Mono | Pre- | | Product | Product | |
|---|---|---|---|---|---|---|---|
| | Peptide | Iso- | cursor | Pre- | Trans- | Transition | |
| Target | Sequence | topic | Charge | cursor | ition | Sequence | Ion |
| Protein | (SEQ ID NO:) | Mass | State | m/z | m/z | (SEQ ID NO:) | Type |
| Cry1Ab | GSAQGIEGSIR (1) | 1074.55 | 2 | 537.78 | 731.4 | GIEGSIR (99) | y7 |
| Cry1Ab | GSAQGIEGSIR | 1074.55 | 2 | 537.78 | 561.3 | EGSIR (100) | y5 |
| Cry1Ab | IVAQLGQGVYR (2) | 1203.68 | 2 | 602.35 | 991.5 | AQLGQGVYR (101) | y9 |
| Cry1Ab | IVAQLGQGVYR | 1203.68 | 2 | 602.35 | 679.4 | GQGVYR (102) | y6 |
| Cry1Ab | TLSSTLYR (3) | 940.51 | 2 | 470.76 | 726.4 | SSTLYR (103) | y6 |
| Cry1Ab | TLSSTLYR | 940.51 | 2 | 470.76 | 639.3 | STLYR (104) | y5 |
| Cry1Ab | DVSVFGQR (4) | 907.46 | 2 | 454.24 | 693.4 | SVFGQR (105) | y6 |
| Cry1Ab | DVSVFGQR | 907.46 | 2 | 454.24 | 507.3 | FGQR (106) | y4 |
| Cry1Ab | TYPIR (5) | 649.37 | 2 | 325.19 | 385.3 | PIR | y3 |
| Cry1Ab | TYPIR | 649.37 | 2 | 325.19 | 265.1 | TY | b2 |
| Cry1Ab | TVSQLTR (6) | 804.46 | 2 | 402.73 | 604.3 | SQLTR (107) | y5 |
| Cry1Ab | TVSQLTR | 804.46 | 2 | 402.73 | 517.3 | QLTR (108) | y4 |
| Cry1Ab | WYNTGLER (7) | 1038.5 | 2 | 519.75 | 689.4 | NTGLER (109) | y6 |
| Cry1Ab | WYNTGLER | 1038.5 | 2 | 519.75 | 852.4 | YNTGLER (110) | y7 |
| Cry1Ab | EWEADPTNPALR (8) | 1398.66 | 2 | 699.84 | 768.4 | PTNPALR (111) | y7 |
| Cry1Ab | EWEADPTNPALR | 1398.66 | 2 | 699.84 | 883.5 | DPTNPALR (112) | y8 |
| Cry1Ab | VWGPDSR (9) | 816.4 | 2 | 408.7 | 531.3 | GPDSR (113) | y5 |
| Cry1Ab | VWGPDSR | 816.4 | 2 | 408.7 | 286.2 | VW | b2 |
| Cry1Ab | APMFSWIHR (10) | 1144.57 | 3 | 382.2 | 312.2 | HR | y2 |
| Cry1Ab | APMFSWIHR | 1144.57 | 3 | 382.2 | 698.4 | SWIHR (114) | y5 |
| Cry1Ab | WGFDAATINSR (11) | 1237.6 | 2 | 619.3 | 661.4 | ATINSR (115) | y6 |
| Cry1Ab | WGFDAATINSR | 1237.6 | 2 | 619.3 | 847.4 | DAATINSR (116) | y8 |
| Cry1Ab | NQAISR (12) | 688.37 | 2 | 344.69 | 446.3 | AISR (117) | y4 |
| Cry1Ab | NQAISR | 688.37 | 2 | 344.69 | 375.2 | ISR | y3 |
| Cry1Ab | IEEFAR (13) | 764.39 | 2 | 382.7 | 522.3 | EFAR (118) | y4 |
| Cry1Ab | IEEFAR | 764.39 | 2 | 382.7 | 651.3 | EEFAR (119) | y5 |
| Cry1Ab | SGFSNSSVSIIR (14) | 1253.65 | 2 | 627.33 | 962.5 | SNSSVSIIR (120) | y9 |
| Cry1Ab | SGFSNSSVSIIR | 1253.65 | 2 | 627.33 | 761.5 | SSVSIIR (121) | y7 |
| Cry1Ab | LSHVSMFR (15) | 976.5 | 2 | 488.76 | 540.3 | SMFR (122) | y4 |
| Cry1Ab | LSHVSMFR | 976.5 | 2 | 488.76 | 639.3 | VSMFR (123) | y5 |
| Cry1Ab | EIYTNPVLENFDGSFR (16) | 1900.91 | 2 | 950.96 | 971.4 | ENFDGSFR (124) | y8 |
| Cry1Ab | EIYTNPVLENFDGSFR | 1900.91 | 2 | 950.96 | 466.2 | GSFR (125) | y4 |
| Cry1Ab | LEGLSNLYQIYAESFR (17) | 1902.96 | 2 | 951.98 | 772.4 | YAESFR (126) | y6 |
| Cry1Ab | LEGLSNLYQIYAESFR | 1902.96 | 2 | 951.98 | 300.2 | LEG | b3 |
| Cry1Ab | YNQFR (18) | 727.35 | 2 | 364.18 | 564.3 | NQFR (127) | y4 |
| Cry1Ab | YNQFR | 727.35 | 2 | 364.18 | 450.2 | QFR | y3 |
| Cry1Ab | YNDLTR (19) | 781.38 | 2 | 391.2 | 504.3 | DLTR (128) | y4 |
| Cry1Ab | YNDLTR | 781.38 | 2 | 391.2 | 618.3 | NDLTR (129) | y5 |
| Cry1Ab | SPHLMDILNSITIYTDAHR | 2197.11 | 3 | 733.04 | 976.5 | TIYTDAHR (130) | y8 |
| | (20) | | | | | | |
| Cry1Ab | SPHLMDILNSITIYTDAHR | 2197.11 | 3 | 733.04 | 762.4 | YTDAHR (131) | y6 |
| Cry1Ab | SAEFNNIIPSSQITQIPLTK | 2201.18 | 2 | 1101.09 | 458.3 | PLTK (132) | y4 |
| | (21) | | | | | | |
| Cry1Ab | SAEFNNIIPSSQITQIPLTK | 2201.18 | 2 | 1101.09 | 889.4 | SAEFNNII (133) | b8 |
| Cry1Ab | QGFSHR (22) | 731.36 | 2 | 366.18 | 546.3 | FSHR (134) | y4 |
| Cry1Ab | QGFSHR | 731.36 | 2 | 366.18 | 603.3 | GFSHR (135) | y5 |
| Cry1Ab | MDNNPNINECIPYNCLSNPEV | 3133.4 | 3 | 1045.14 | 759.4 | EVLGGER (136) | y7 |
| | EVLGGER (23) | | | | | | |
| Cry1Ab | MDNNPNINECIPYNCLSNPEV | 3133.4 | 3 | 1045.14 | 418.2 | GGER (137) | y4 |
| | EVLGGER | | | | | | |
| Cry1Ab | ELTLTVLDIVSLFPNYDSR | 2195.16 | 2 | 1098.08 | 898.4 | FPNYDSR (138) | y7 |
| | (24) | | | | | | |
| Cry1Ab | ELTLTVLDIVSLFPNYDSR | 2195.16 | 2 | 1098.08 | 751.3 | PNYDSR (139) | y6 |
| Cry1Ab | RPFNIGINNQQLSVLDGTEFA | 3728.87 | 3 | 1243.63 | 692.4 | PSAVYR (140) | y6 |
| | YGTSSNLPSAVYR (25) | | | | | | |
| Cry1Ab | RPFNIGINNQQLSVLDGTEFA | 3728.87 | 3 | 1243.63 | 338.2 | YR | y2 |
| | YGTSSNLPSAVYR | | | | | | |
| Cry1Ab | SGTVDSLDEIPPQNNNVPPR | 2149.05 | 2 | 1075.03 | 369.2 | PPR | y3 |
| | (26) | | | | | | |
| Cry1Ab | SGTVDSLDEIPPQNNNVPPR | 2149.05 | 2 | 1075.03 | 904.4 | SGTVDSLDE (141) | b9 |
| eCry3.1Ab | TDVTDYHIDQV (27) | 1305.6 | 2 | 653.3 | 990.5 | TDYHIDQV (142) | y8 |
| eCry3.1Ab | TDVTDYHIDQV | 1305.6 | 2 | 653.3 | 889.4 | DYHIDQV (143) | y7 |
| eCry3.1Ab | AVNELFTSSNQIGLK (28) | 1620.86 | 2 | 810.93 | 947.5 | TSSNQIGLK (144) | y9 |
| eCry3.1Ab | AVNELFTSSNQIGLK | 1620.86 | 2 | 810.93 | 846.5 | SSNQIGLK (145) | y8 |
| eCry3.1Ab | ITQLPLTK (29) | 913.57 | 2 | 457.29 | 699.4 | QLPLTK (146) | y6 |
| eCry3.1Ab | ITQLPLTK | 913.57 | 2 | 457.29 | 800.5 | TQLPLTK (147) | y7 |
| eCry3.1Ab | GLDSSTTK (30) | 808.4 | 2 | 404.71 | 638.3 | DSSTTK (148) | y6 |
| eCry3.1Ab | GLDSSTTK | 808.4 | 2 | 404.71 | 523.3 | SSTTK (149) | y5 |
| eCry3.1Ab | QCAGIRPYDGR (31) | 1235.6 | 3 | 412.54 | 607.3 | PYDGR (150) | y5 |
| eCry3.1Ab | QCAGIRPYDGR | 1235.6 | 3 | 412.54 | 347.2 | DGR | y3 |
| eCry3.1Ab | IEFVPAEVTFEAEYDLER | 2157.04 | 2 | 1079.02 | 390.2 | IEF | b3 |
| | (32) | | | | | | |
| eCry3.1Ab | IEFVPAEVTFEAEYDLER | 2157.04 | 2 | 1079.02 | 417.2 | LER | y3 |
| mCry3A | LQSGASVVAGPR (252) | 1141.63 | 2 | 571.32 | 900.5 | SGASVVAGPR | y10 |
| | | | | | | (253) | |
| mCry3A | LQSGASVVAGPR | 1141.63 | 2 | 571.32 | 685.4 | SVVAGPR (254) | y7 |
| mCry3A | ITQLPLVK (33) | 911.59 | 2 | 456.3 | 697.5 | QLPLVK (151) | y6 |
| mCry3A | ITQLPLVK | 911.59 | 2 | 456.3 | 798.5 | TQLPLVK (152) | y7 |
| mCry3A | MTADNNTEALDSSTTK (34) | 1698.75 | 2 | 849.88 | 822.4 | ALDSSTTK (153) | y8 |
| mCry3A | MTADNNTEALDSSTTK | 1698.75 | 2 | 849.88 | 951.5 | EALDSSTTK (154) | y9 |
| mCry3A | VYIDK (35) | 637.36 | 2 | 319.18 | 538.3 | YIDK (155) | y4 |
| mCry3A | VYIDK | 637.36 | 2 | 319.18 | 375.2 | IDK | y3 |
| Vip3Aa20 | DGGISQFIGDK (36) | 1136.56 | 2 | 568.78 | 794.4 | SQFIGDK (156) | y7 |
| Vip3Aa20 | DGGISQFIGDK | 1136.56 | 2 | 568.78 | 319.2 | GDK | y3 |
| Vip3Aa20 | LITLTCK (37) | 791.47 | 2 | 396.24 | 565.3 | TLTCK (157) | y5 |
| Vip3Aa20 | LITLTCK | 791.47 | 2 | 396.24 | 351.2 | TCK | y3 |
| Vip3Aa20 | ELLLATDLSNK (38) | 1216.68 | 2 | 608.84 | 748.4 | ATDLSNK (158) | y7 |
| Vip3Aa20 | ELLLATDLSNK | 1216.68 | 2 | 608.84 | 861.5 | LATDLSNK (159) | y8 |
| Vip3Aa20 | FTTGTDLK (255) | 882.46 | 2 | 441.73 | 634.3 | TGTDLK (256) | y6 |
| Vip3Aa20 | FTTGTDLK | 882.46 | 2 | 441.73 | 260.2 | LK | y2 |
| Vip3Aa20 | FEELTFATETSSK (39) | 1489.71 | 2 | 745.36 | 971.5 | TFATETSSK (160) | y9 |
| Vip3Aa20 | FEELTFATETSSK | 1489.71 | 2 | 745.36 | 870.4 | FATETSSK (161) | y8 |
| Vip3Aa20 | EVLFEK (40) | 764.42 | 2 | 382.71 | 423.2 | FEK | y3 |
| Vip3Aa20 | EVLFEK | 764.42 | 2 | 382.71 | 536.3 | LFEK (162) | y4 |
| Vip3Aa20 | TASELITK (41) | 862.49 | 2 | 431.75 | 690.4 | SELITK (163) | y6 |
| Vip3Aa20 | TASELITK | 862.49 | 2 | 431.75 | 761.4 | ASELTIK (164) | y7 |
| Vip3Aa20 | DVSEMFTTK (42) | 1057.49 | 2 | 529.25 | 843.4 | SEMFTTK (165) | y7 |
| Vip3Aa20 | DVSEMFTTK | 1057.49 | 2 | 720.71 | 284.2 | DVS | b3 |
| Vip3Aa20 | LLGLADIDYTSIMNEHLNK | 2160.1 | 3 | 720.71 | 885.4 | MNEHLNK (166) | y7 |
| | (43) | | | | | | |
| Vip3Aa20 | LLGLADIDYTSIMNEHLNK | 2160.1 | 3 | 720.71 | 885.4 | MNEHLNK (167) | y7 |
| Vip3Aa20 | IDFTK (44) | 623.34 | 2 | 312.17 | 510.3 | DFTK (168) | y4 |
| Vip3Aa20 | IDFTK | 623.34 | 2 | 312.17 | 395.2 | FTK | y3 |
| Vip3Aa20 | TDTGGDLTLDEILK (45) | 1490.76 | 2 | 745.88 | 831.5 | TLDEILK (169) | y7 |
| Vip3Aa20 | TDTGGDLTLDEILK | 1490.76 | 2 | 745.88 | 944.6 | LTLDEILK (170) | y8 |
| Vip3Aa20 | DIMNMIFK (46) | 1011.5 | 2 | 506.25 | 783.4 | MNMIFK (171) | y6 |
| Vip3Aa20 | DIMNMIFK | 1011.5 | 2 | 506.25 | 652.3 | NMIFK (172) | y5 |
| Vip3Aa20 | ALYVHK (47) | 730.42 | 2 | 365.72 | 546.3 | YVHK (173) | y4 |
| Vip3Aa20 | ALYVHK | 730.42 | 2 | 365.72 | 284.2 | HK | y2 |
| Vip3Aa20 | VNILPTLSNTFSNPNYAK | 1993.04 | 2 | 997.02 | 327.2 | VNI | b3 |
| | (48) | | | | | | |
| Vip3Aa20 | VNILPTLSNTFSNPNYAK | 1993.04 | 2 | 997.02 | 440.3 | VNIL (174) | b4 |
| Vip3Aa20 | ITSMLSDVIK (49) | 1106.61 | 2 | 553.81 | 892.5 | SMLSDVIK (175) | y8 |
| Vip3Aa20 | ITSMLSDVIK | 1106.61 | 2 | 553.81 | 993.5 | TSMLSDVIK (176) | y9 |
| Vip3Aa20 | QNLQLDSFSTYR (50) | 1471.72 | 2 | 736.36 | 875.4 | DSFSTYR (177) | y7 |
| Vip3Aa20 | QNLQLDSFSTYR | 1471.72 | 2 | 736.36 | 988.5 | LDSFSTYR (178) | y8 |
| Vip3Aa20 | DSLSEVIYGDMDK (51) | 1471.66 | 2 | 736.33 | 841.4 | IYGDMDK (179) | y7 |
| Vip3Aa20 | DSLSEVIYGDMDK | 1471.66 | 2 | 736.33 | 940.4 | VIYGDMDK (180) | y8 |
| Vip3Aa20 | MIVEAKPGHALIGFEISNDSI | 2682.45 | 3 | 894.82 | 976.5 | SNDSITVLK (181) | y9 |
| | TVLK (52) | | | | | | |
| Vip3Aa20 | MIVEAKPGHALIGFEISNDSI | 2682.45 | 3 | 894.82 | 344.2 | MIV | b3 |
| | TVLK | | | | | | |
| Vip3Aa20 | VYFSVSGDANVR (53) | 1313.65 | 2 | 657.33 | 718.3 | SGDANVR (182) | y7 |
| Vip3Aa20 | VYFSVSGDANVR | 1313.65 | 2 | 657.33 | 904.4 | SVSGDANVR (183) | y9 |
| Vip3Aa20 | NQQLLNDISGK (54) | 1229.65 | 2 | 615.33 | 859.5 | LLNDISGK (184) | y8 |
| Vip3Aa20 | NQQLLNDISGK | 1229.65 | 2 | 615.33 | 746.4 | LNDISGK (185) | y7 |
| Vip3Aa20 | VESSEAEYR (55) | 1069.48 | 2 | 535.24 | 841.4 | SSEAEYR (186) | y7 |
| Vip3Aa20 | VESSEAEYR | 1069.48 | 2 | 535.24 | 970.4 | ESSEAEYR (187) | y8 |
| Vip3Aa20 | YMSGAK (56) | 656.31 | 2 | 328.66 | 362.2 | SGAK (188) | y3 |
| Vip3Aa20 | YMSGAK | 656.31 | 2 | 328.66 | 493.2 | MSGAK (189) | y5 |
| Vip3Aa20 | DGSPADILDELTELTELAK | 2030.02 | 2 | 1015.51 | 904.5 | TELTELAK (190) | y8 |
| | (57) | | | | | | |
| Vip3Aa20 | DGSPADILDELTELTELAK | 2030.02 | 2 | 1015.51 | 656.3 | DGSPADI (191) | b7 |
| Vip3Aa20 | VYEAK (58) | 609.32 | 2 | 305.17 | 510.3 | YEAK (192) | y4 |
| Vip3Aa20 | VYEAK | 609.32 | 2 | 305.17 | 347.2 | EAK | y3 |
| Vip3Aa20 | LDAINTMLR (59) | 1046.57 | 2 | 523.79 | 634.3 | NTMLR (193) | y5 |
| Vip3Aa20 | LDAINTMLR | 1046.57 | 2 | 523.79 | 818.5 | AINTMLR (194) | y7 |
| Vip3Aa20 | GKPSIHLK (60) | 879.54 | 2 | 440.27 | 694.4 | PSIHLK (195) | y6 |
| Vip3Aa20 | GKPSIHLK | 879.54 | 2 | 440.27 | 397.3 | HLK | y3 |
| Vip3Aa20 | DENTGYIHYEDTNNNLEDYQT | 2903.26 | 3 | 968.43 | 881.4 | DYQTINK (196) | y7 |
| | INK (61) | | | | | | |
| Vip3Aa20 | DENTGYIHYEDTNNNLEDYQT | 2903.26 | 3 | 968.43 | 261.2 | NK | y2 |
| | INK | | | | | | |
| Vip3Aa20 | DNFYIELSQGNNLYGGPIVHF | 3102.52 | 3 | 1034.85 | 377.1 | DNF | b3 |
| | YDVSIK (62) | | | | | | |
| Vip3Aa20 | DNFYIELSQGNNLYGGPIVHF | 3102.52 | 3 | 1034.85 | 540.2 | DNFY (197) | b4 |
| | YDVSIK | | | | | | |
| Vip3Aa20 | LLCPDQSEQIYYTNNIVFPNE | 3104.53 | 3 | 1035.51 | 963.5 | PNEYVITK (198) | y8 |
| | YVITK (63) | | | | | | |
| Vip3Aa20 | LLCPDQSEQIYYTNNIVFPNE | 3104.53 | 3 | 1035.51 | 330.2 | LLC | b3 |
| | YVITK | | | | | | |
| Vip3Aa20 | SQNGDEAWGDNFIILEISPSE | 2449.15 | 2 | 1225.08 | 547.3 | SPSEK (199) | y5 |
| | K (64) | | | | | | |
| Vip3Aa20 | SQNGDEAWGDNFIILEISPSE | 2449.15 | 2 | 1225.08 | 902.5 | LEISPSEK (200) | y8 |
| | K | | | | | | |
| Vip3Aa20 | NAYVDHTGGVNGTK (65) | 1432.68 | 2 | 716.84 | 349.2 | NAY | b3 |
| Vip3Aa20 | NAYVDHTGGVNGTK | 1432.68 | 2 | 716.84 | 985.5 | DHTGGVNSGTK | y11 |
| | | | | | | (201) | |
| Vip3Aa20 | LDGVNGSLNDLIAQGNLNTEL | 2385.23 | 2 | 1193.12 | 975.5 | GNLNTELSK (202) | y9 |
| | SK (66) | | | | | | |
| Vip3Aa20 | LDGVNGSLNDLIAQGNLNTEL | 2385.23 | 2 | 1193.12 | 691.4 | NTELSK (203) | y6 |
| | SK | | | | | | |
| Vip3Aa20 | IANEQNQVLNDVNNK (67) | 1712.86 | 2 | 856.93 | 816.4 | LNDVNNK (204) | y7 |
| Vip3Aa20 | IANEQNQVLNDVNNK | 1712.86 | 2 | 856.93 | 703.3 | NDVNNK (205) | y6 |
| Vip3Aa20 | YEVTANFYDSSTGEIDLNK | 2165.99 | 2 | 1083.5 | 293.1 | YE | b2 |
| | (68) | | | | | | |
| Vip3Aa20 | YEVTANFYDSSTGEIDLNK | 2165.99 | 2 | 1083.5 | 489.3 | DLNK (206) | y4 |
| Vip3Aa20 | QNYALSLQIEYLSK (69) | 1669.88 | 2 | 835.44 | 880.5 | QIEYLSK (207) | y7 |
| Vip3Aa20 | QNYALSLQIEYLSK | 1669.88 | 2 | 835.44 | 993.6 | LQIEYLSK (208) | y8 |
| Vip3Aa20 | QLQEISDK (70) | 960.45 | 2 | 480.75 | 349.2 | SDK | y3 |
| Vip3Aa20 | QLQEISDK | 960.45 | 2 | 480.75 | 719.4 | QEISDK (209) | y6 |
| Vip3Aa20 | LLSPELINTNNWTSTGSTNIS | 3422.72 | 3 | 1141.58 | 580.3 | YQGGR (210) | y5 |
| | GNTLTLYQGGR (71) | | | | | | |
| Vip3Aa20 | LLSPELINTNNWTSTGSTNIS | 3422.72 | 3 | 1141.58 | 794.4 | TLYQGGR (211) | y7 |
| | GNTLTLYQGGR | | | | | | |
| Vip3Aa20 | YVNEK (72) | 652.33 | 2 | 326.67 | 390.2 | NEK | y3 |
| Vip3Aa20 | YVNEK | 652.33 | 2 | 326.67 | 489.3 | VNEK (212) | y4 |
| Vip3Aa20 | QNYQVDK (73) | 894.43 | 2 | 447.72 | 262.1 | DK | y2 |
| Vip3Aa20 | QNYQVDK | 894.43 | 2 | 447.72 | 361.2 | VDK | y3 |
| dmEPSPS | MAGAEEIVLQPIK (74) | 1398.77 | 2 | 699.89 | 357.3 | PIK | y3 |
| dmEPSPS | MAGAEEIVLQPIK | 1398.77 | 2 | 699.89 | 939.6 | EIVLQPIK (213) | y8 |
| dmEPSPS | SLTAAVTAAGGNATYVLDGV | 2104.1 | 2 | 1052.56 | 272.2 | PR | y2 |
| | PR (257) | | | | | | |
| dmEPSPS | SLTAAVTAAGGNATYVLDGV | 2104.1 | 2 | 1052.56 | 428.3 | GVPR (258) | y4 |
| | PR | | | | | | |
| dmEPSPS | FPVEDAK (75) | 805.41 | 2 | 403.21 | 658.3 | PVEDAK (214) | y6 |
| dmEPSPS | FPVEDAK | 805.41 | 2 | 403.21 | 561.3 | VEDAK (215) | y5 |
| dmEPSPS | EISGTVK (76) | 733.41 | 2 | 367.21 | 491.3 | SGTVK (216) | y5 |
| dmEPSPS | EISGTVK | 733.41 | 2 | 367.21 | 404.3 | GTVK (217) | y4 |
| dmEPSPS | ILLLAALSEGTTVVDNLLNSE | 3340.78 | 3 | 1114.27 | 595.4 | ILLLAA (218) | b6 |
| | DVHYMLGALR (77) | | | | | | |
| dmEPSPS | ILLLAALSEGTTVVDNLLNSE | 3340.78 | 3 | 1114.27 | 960.5 | HYMLGALR (219) | y8 |
| | DVHYMLGALR | | | | | | |
| PAT | DFELPAPPRPVRPVTQI | 1932.07 | 3 | 644.7 | 392.1 | DFE | b3 |
| | (78) | | | | | | |
| PAT | DFELPAPPRPVRPVTQI | 1932.07 | 3 | 644.7 | 263.1 | DF | b2 |
| PAT | LGLGSTLYTHLLK (79) | 1415.83 | 3 | 472.61 | 774.5 | YTHLLK (220) | y6 |
| PAT | LGLGSTLYTHLLK | 1415.83 | 3 | 472.61 | 611.4 | THLLK (221) | y5 |
| PAT | MSPER (80) | 619.29 | 2 | 310.15 | 401.2 | PER | y3 |
| PAT | MSPER | 619.29 | 2 | 310.15 | 488.2 | SPER (222) | y4 |
| PAT | HGGWHDVGFWQR (81) | 1481.68 | 3 | 494.57 | 693.3 | GFWQR (223) | y5 |
| PAT | HGGWHDVGFWQR | 1481.68 | 3 | 494.57 | 792.4 | VGFWQR (224) | y6 |
| PAT | NAYDWTVESTVYVSHR | 1926.9 | 3 | 642.97 | 948.5 | STVYVSHR (225) | y8 |
| | (82) | | | | | | |
| PAT | NAYDWTVESTVYVSHR | 1926.9 | 3 | 642.97 | 399.2 | SHR | y3 |
| PAT | TEPQTPQEWIDDLER (83) | 1856.87 | 2 | 928.94 | 557.3 | TEPQT (226) | b5 |
| PAT | TEPQTPQEWIDDLER | 1856.87 | 2 | 928.94 | 532.3 | DLER (227) | y4 |
| PAT | AAGYK (84) | 509.27 | 2 | 255.14 | 367.2 | GYK | y3 |
| PAT | AAGYK | 509.27 | 2 | 255.14 | 438.2 | AGYK (228) | y4 |
| PAT | YPWLVAEVEGVVAGIAYAGPW | 2375.24 | 2 | 1188.13 | 487.3 | GPWK (229) | y4 |
| | K (85) | | | | | | |
| PAT | YPWLVAEVEGVVAGIAYAGPW | 2375.24 | 2 | 1188.13 | 962.5 | GIAYAGPWK (230) | y9 |
| | K | | | | | | |
| PAT | RPVEIRPATAADMAAVCDIVN | 3559.78 | 3 | 1187.26 | 824.4 | TSTVNFR (231) | y7 |
| | HYIETSTVNFR (86) | | | | | | |
| PAT | RPVEIRPATAADMAAVCDIVN | 3559.78 | 3 | 1187.26 | 436.2 | NFR | y3 |
| | HYIETSTVNFR | | | | | | |
| PMI | ENAAGIPMDAAER (87) | 1344.62 | 2 | 672.81 | 789.4 | PMDAAER (232) | y7 |
| PMI | ENAAGIPMDAAER | 1344.62 | 2 | 672.81 | 959.5 | GIPMDAAER (233) | y9 |
| PMI | ALAILK (88) | 628.44 | 2 | 314.72 | 444.3 | AILK (234) | y4 |
| PMI | ALAILK | 628.44 | 2 | 314.72 | 260.2 | LK | y2 |
| PMI | SALDSQQGEPWQTIR (89) | 1715.83 | 2 | 858.42 | 800.4 | PWQTIR (235) | y6 |
| PMI | SALDSQQGEPWQTIR | 1715.83 | 2 | 858.42 | 986.5 | GEPWQTIR (236) | y8 |
| PMI | GSQQLQLKPGESAFIAANESP | 2599.37 | 3 | 867.13 | 944.5 | ANESPVTVK (237) | y9 |
| | VTVK (90) | | | | | | |
| PMI | GSQQLQLKPGESAFIAANESP | 2599.37 | 3 | 867.13 | 543.4 | PVTVK (238) | y5 |
| | VTVK | | | | | | |
| PMI | FEAKPANQLLTQPVK (91) | 1683.94 | 3 | 561.99 | 685.4 | LTQPVK (239) | y6 |
| PMI | FEAKPANQLLTQPVK | 1683.94 | 3 | 561.99 | 343.2 | PVK | y3 |
| PMI | STLLGEAVAK (92) | 988.57 | 2 | 494.79 | 574.3 | GEAVAK (240) | y6 |
| PMI | STLLGEAVAK | 988.57 | 2 | 494.79 | 687.4 | LGEAVAK (241) | y7 |
| PMI | LINSVQNYAWGSK (93) | 1479.76 | 2 | 740.38 | 953.4 | QNYAWGSK (242) | y8 |
| PMI | LINSVQNYAWGSK | 1479.76 | 2 | 740.38 | 825.4 | NYAWGSK (243) | y7 |
| PMI | HNSEIGFAK (94) | 1002.5 | 2 | 501.75 | 865.4 | NSEIGFAK (244) | y8 |
| PMI | HNSEIGFAK | 1002.5 | 2 | 501.75 | 252.1 | HN | b2 |
| PMI | VLCAAQPLSIQVHPNK (95) | 1717.94 | 2 | 859.47 | 586.3 | VLCAAQ (245) | b6 |
| PMI | VLCAAQPLSIQVHPNK | 1717.94 | 2 | 859.47 | 358.2 | PNK | y3 |
| PMI | TALTELYGMENPSSQPMAELW | 2989.39 | 3 | 997.13 | 826.4 | WMGAHPK (246) | y7 |
| | MGAHPK (96) | | | | | | |
| PMI | TALTELYGMENPSSQPMAELW | 2989.39 | 3 | 997.13 | 516.3 | TALTE (247) | b5 |
| | MGAHPK | | | | | | |
| PMI | LSELFASLLNMQGEEK (97) | 2808.91 | 3 | 904.96 | 835.4 | NMQGEEK (248) | y7 |
| PMI | LSELFASLLNMQGEEK | 2808.91 | 3 | 904.96 | 948.4 | LNMQGEEK (249) | y8 |
| PMI | QGAELDFPIPVDDFAFSLHDL | 2676.28 | 3 | 892.77 | 914.5 | SLHDLSDK (250) | y8 |
| | SDK (98) | | | | | | |
| PMI | QGAELDFPIPVDDFAFSLHDL | 2676.28 | 3 | 892.77 | 714.3 | HDLSDK (251) | y6 |
| | SDK | | | | | | |

Following the identification of multiple potential surrogate peptides for the target proteins Cry1Ab, eCry3.1Ab, mCry3A, Vip3A, dmEPSPS, PAT and PMI, individual surrogate peptides were further selected based upon transition ions that provide optimal signal intensity and have the ability to discriminate the target surrogate peptide from other species present in the biological sample matrix (for example, maize leaf, root, pollen, or kernel (seed)). This includes both matrix interferences (i.e. matrix interferences are one or more specific constituents within the matrix that are detected at or near the peptide of interest) and potential carry-over (i.e. carry-over is a result of previously injected samples that elute upon subsequent analyses due to chemical/physical characteristics of the sample analysis system or both). These optimized transitions of a cassette containing the individual surrogate peptides make up the overall MRM assay. In the present disclosure those surrogate peptides from Cry1Ab, eCry3.1Ab, mCry3A, Vip3A, dmEPSPS, PAT and PMI that provided the highest-sensitivity (most intense fragments) and that had the desired specificity were further selected to make up a cassette of surrogate peptides for quantifying the seven targeted proteins. Table 2 lists preferred surrogate peptides for each target protein Cry1Ab, eCry3.1Ab, mCry3A, Vip3Aa20 dmEPSPS, PAT and PMI, and the corresponding stable-isotope labelled (SIL) peptide. The cassette of surrogate peptides comprises of one or more of the peptides to be monitored and/or quantified simultaneously. This cassette of surrogate peptides with the specific fragmentation/transition ions for each peptide may be used in a MRM assay to quantify the corresponding target proteins.

TABLE 2
Surrogate peptides and SIL peptides that
specifically detect target proteins.

| | Surrogate | SIL Surrogate |
|---|---|---|
| Target | Peptide | Peptide |
| Protein | (SEQ ID NO:) | [Heavy Amino Acid] |
| Cry1Ab | SAEFNNIIPSSQITQ | SAEFNNIIPSSQITQ |
| | IPLTK | IPLTK |
| | (SEQ ID NO: 21) | [C13N15-K] |
| eCry3.1Ab | TDVTDYHIDQV | TDVTDYHIDQV |
| | (SEQ ID NO: 27) | [C13N15-V] |
| mCry3A | LQSGASVVAGPR | LQSGASVVAGPR |
| | (SEQ ID NO: 252) | [C13N15-R] |
| Vip3A | FTTGTDLK | FTTGTDLK |
| | (SEQ ID NO: 255) | [C13N15-K] |
| dmEPSPS | SLTAAVTAAGGNATYV | SLTAAVTAAGGNATYV |
| | LDGVPR | LDGVPR |
| | (SEQ ID NO: 257) | [C13N15-R] |
| PAT | LGLGSTLYHLLK | LGLGSTLYHLLK |
| | (SEQ ID NO: 79) | [C13N15-K] |
| PMI | SALDSQQGEPWQTIR | SALDSQQGEPWQTIR |
| | (SEQ ID NO: 89) | [C13N15-R] |

### Example 2—Assay for Detection of Transgenic Proteins in Transgenic Plant Tissues

The development of sensitive methods for directly monitoring target proteins is highly desirable for quantitative assessments in biological matrices, such as from tissues of transgenic plants, e.g. leaf, kernel, root and pollen tissue. Multiple reaction monitoring (MRM) mass spectrometry has emerged as a promising platform to quantify multiple proteins within a given sample by liquid chromatography (LC) coupled with tandem mass spectrometry (MS/MS/). MRM assays utilize sequence-specific tandem MS fragmentations of proteolytic peptides, thereby providing highly selective and specific measurements for distinct target proteins. Despite these advances, it remains challenging to obtain accurate quantitative measurements on low abundant proteins or those that have specific physicochemical properties which impacts separation.

MRM assays typically are performed on a triple quadrupole mass spectrometer, although this methodology may also be applied in an ion trap instrument where, upon fragmentation of a signature ion, MS/MS data are acquired on a fragment ion in a defined mass range or on a full mass range. A series of transitions (signature/fragment ion m/z pairs) in combination with the retention time of the targeted peptide can constitute an MRM assay. To achieve an optimum MRM assay (1) the target protein/peptide needs to be selected; (2) the surrogate peptides must generate good MS and MS/MS signals; (3) each selected peptide fragment ions must provide optimal signal intensity and distinguish the target peptide from other peptide species present in the complex biological sample. Collectively, the surrogate peptide and fragment ions provide high specificity for peptide selections since only desired transitions are recorded and other signals present in the sample are ignored.

A common misperception in the art is that MRM assays guarantee specificity and sensitivity, sample preparation may be simplified and even eliminated, and no or very little chromatographic separation is required. However, contrary to this incorrect perception, MRM assays tend to be highly impacted by the complexity of the sample, thus reducing the sensitivity of specific target peptides. The specificity and sensitivity may be influenced by matrix effects, e.g. differences between leaf, pollen, root, stem, and result in ion suppression which occurs during MS analysis. In general, most charged or ionisable molecules, e.g. salts, chaotropes, detergents, polymers, all nonvolatile ionic compounds, interfere with ionization of the desired analyte, i.e. peptide/protein, thus competing and causing signal suppression and/or elevated background noise. Ion suppression negatively affects several analytical parameters, such as detection capability, precision and accuracy. Thus, to overcome all of these deficiencies in the MRM mass spectrometry methods in the art, there is a need to develop a method for efficient extraction of target proteins from complex biological samples, e.g. transgenic plant samples, to enrich the target proteins and/or remove interferences that may reduce the ion intensity of the targeted protein/peptide and affect reproducibility and accuracy of the assay.

In general, improving the sample preparation may be the most effective way of reducing matrix effects and circumventing ion suppression. The method enables the ability to enrich for selected target proteins and peptides without concentrating the interferences allowing for accurate and precise quantitation at low target protein concentrations.

A MRM-based assay utilizing the cassette of surrogate peptides (from either Table 1 or Table 2) was used to measure Cry1Ab, eCry3.1Ab, mCry3A, Vip3A, dmEPSPS, PAT and PMI in different transgenic events containing at least one of the seven proteins (Table 4). The transgenic events evaluated in the study were as follows: Bt11 (Cry1Ab and PAT); 5307 (eCry3.1Ab and PMI); MIR604 (mCry3A and PMI); MIR162 (Vip3Aa20 and PMI) and GA21 (dmEPSPS).

Tissue extraction—12-15 mg lyophilized tissue (leaf, root, pollen kernel and whole plant) is placed into 2 μL Lysing Matrix A FastPrep tube (MP Biomedicals, Santa Ana, Calif.). 1.0-1.5 μL (w/v) of PBS with 0.1% RapiGest is then added. Samples are then extracted in a FastPrep-24 tissue homogenizer (MP Biomedicals, Santa Ana, Calif.) with Lysing Matrix A (garnet matrix and ¼″ ceramic sphere beads) for 1 cycle (40 s, speed setting 6) at ambient temperature. Proteins are extracted from the selected tissue in 50 μl extraction buffer (6M urea, 2M thiourea, 5 mM EDTA, 0.1M HEPES) per mg lyophilized tissue

Centrifugation—After tissue extraction, the samples are centrifuged at 4° C. at 15,000 g for about 5 min. This step pulls out insoluble proteins, e.g. histones and actin, thus reducing the complexity of the extract prior to digestion. Therefore, only soluble proteins move to the enzyme digestion step.

Trypsin Digestion—Total protein concentration of the supernatant from the centrifugation step is adjusted to about 0.2 μg/μl by dilution in homogenization buffer. The equivalent of 30 μg of protein is transferred to a well plate. One volume of trifluoroethanol is added to the samples and incubated for about 30 min at room temperature while shaking at low speed. Four volumes of 100 mM ammonium bicarbonate is added. About 12 μl of trypsin (0.1 μg/μl) is then added. Samples are incubated overnight at 37° C. Samples are then quenched with 20% formic acid (1% final). 20 μl of stable isotope-labelled peptide is then added.

Centrifugation—Samples from the previous step are then centrifuged at 4° C. at 15,000 g for about 5 min.

Desalt by MCX—After centrifugation, the samples are desalted. This step is performed on an ion exchange column. This desalting step concentrates the peptides of interest by discarding peptides that are not of interest in the wash-through. In addition to removing peptides that are not of interest, this step also removes salts and small molecules that may interfere with the ionization and detection of the surrogate peptides of interest. Concentrating the peptides of interest and removing interfering salts and small molecules increases the sensitivity of the MRM assay of the invention over other methods known in the art.

QTRAP-MRM—MRM analysis is performed using a QTRAP 6500 coupled to a NanoAcquity UPLC with a Halo Peptide ES-C18 column. The flow rate is about 18 μl/min. Solvent A is about 97/3 water/DMSO+0.2% formic acid (FA) and Solvent B is about 97/3 acetonitrile (CAN)/DMSO+0.2% FA. The autosampler temperature is kept at about 4° C. during analysis. A total of 8 μl of sample is injected onto the column maintained at ambient temperature.

Data Analysis—data acquisition is performed using Analyst software (AB SCIEX, Ontario, Canada) and data analysis using Multiquant software (AB SCIEX).

To determine levels of detection (LOD) of target transgenic proteins using the preferred labelled surrogate peptides of the invention, all seven target proteins, Cry1Ab, eCry3.1Ab, mCry3A, Vip3A, dmEPSPS, PAT and PMI, were mixed together and added to leaf, root, kernel and pollen tissue of non-transgenic corn plants. Tables 3-6 show the level of detection (LOD) of target proteins by the MRM and demonstrates that each labelled surrogate peptide and its resulting transition ions are capable of selectively detecting and quantitating a target protein when the target protein is in the presence of other transgenic and non-transgenic proteins across all plant matrices. Good linearity (r=0.988-0.998) was achieved for each preferred surrogate peptide of Table 2. LODs for each surrogate peptide were below the quantitative range established by ELISA indicating that the compositions and methods of the invention are equal to or better than the current standard used to quantitate transgenic proteins in plants.

TABLE 3
LOD of target proteins in corn leaf matrix. (LOD = fmol/μg total protein)

| Target | Labelled Surrogate Peptide | | | | | | |
|---|---|---|---|---|---|---|---|
| Protein | CrylAb | eCry3.1Ab | mCry3A | Vip3A | dmEPSPS | PAT | PMI |
| CrylAb | 0.050 | nd | nd | nd | nd | nd | nd |
| eCry3.1Ab | nd | 0.125 | nd | nd | nd | nd | nd |
| mCry3A | nd | nd | 0.125 | nd | nd | nd | nd |
| Vip3A | nd | nd | nd | 0.025 | nd | nd | nd |
| dmEPSPS | nd | nd | nd | nd | 1.250 | nd | nd |
| PAT | nd | nd | nd | nd | nd | 0.025 | nd |
| PMI | nd | nd | nd | nd | nd | nd | 0.050 |

TABLE 4
LOD of target proteins in corn kernel matrix. (LOD = fmol/μg total protein)

| Target | Labelled Surrogate Peptide | | | | | | |
|---|---|---|---|---|---|---|---|
| Protein | CrylAb | eCry3.1Ab | mCry3A | Vip3A | dmEPSPS | PAT | PMI |
| CrylAb | 0.050 | nd | nd | nd | nd | nd | nd |
| eCry3.1Ab | nd | 0.250 | nd | nd | nd | nd | nd |
| mCry3A | nd | nd | 0.125 | nd | nd | nd | nd |
| Vip3A | nd | nd | nd | 0.020 | nd | nd | nd |
| dmEPSPS | nd | nd | nd | nd | 2.500 | nd | nd |
| PAT | nd | nd | nd | nd | nd | 0.050 | nd |
| PMI | nd | nd | nd | nd | nd | nd | 0.100 |

TABLE 5
LOD of target proteins in corn root matrix. (LOD = fmol/μg total protein)

| Target | Labelled Surrogate Peptide | | | | | | |
|---|---|---|---|---|---|---|---|
| Protein | CrylAb | eCry3.1Ab | mCry3A | Vip3A | dmEPSPS | PAT | PMI |
| CrylAb | 0.050 | nd | nd | nd | nd | nd | nd |
| eCry3.1Ab | nd | 0.125 | nd | nd | nd | nd | nd |
| mCry3A | nd | nd | 0.125 | nd | nd | nd | nd |
| Vip3A | nd | nd | nd | 0.025 | nd | nd | nd |
| dmEPSPS | nd | nd | nd | nd | 1.250 | nd | nd |
| PAT | nd | nd | nd | nd | nd | 0.050 | nd |
| PMI | nd | nd | nd | nd | nd | nd | 0.025 |

TABLE 6
LOD of target proteins in corn pollen matrix. (LOD = fmol/μg total protein)

| Target | Labelled Surrogate Peptide | | | | | | |
|---|---|---|---|---|---|---|---|
| Protein | CrylAb | eCry3.1Ab | mCry3A | Vip3A | dmEPSPS | PAT | PMI |
| CrylAb | 0.050 | nd | nd | nd | nd | nd | nd |
| eCry3.1Ab | nd | 0.250 | nd | nd | nd | nd | nd |
| mCry3A | nd | nd | 0.125 | nd | nd | nd | nd |
| Vip3A | nd | nd | nd | 0.025 | nd | nd | nd |
| dmEPSPS | nd | nd | nd | nd | 2.500 | nd | nd |
| PAT | nd | nd | nd | nd | nd | 0.050 | nd |
| PMI | nd | nd | nd | nd | nd | nd | 0.050 |

The preferred labelled surrogate peptides (Table 2) and their transition ions were then tested to determine their ability to specifically detect a target protein in leaf, kernel, root and pollen tissue from a transgenic corn plant comprising a transgenic event selected form the group consisting of Bt11 (comprises Cry1Ab and PAT), 5307 (comprises eCry3.1Ab and PMI), MIR604 (comprises mCry3A and PMI), MIR162 (comprises Vip3A and PMI) and GA21 (comprises dmEPSPS). Each of the seven preferred surrogate peptides were tested against each of the transgenic events. Table 7 shows the results of the quantitation of the target proteins. The results demonstrate that the Cry1Ab and PAT surrogate peptide and labeled surrogate peptide are able to detect and/or quantitate Cry1Ab and PAT in leaf, kernel, root and pollen from a transgenic corn plant comprising event Bt11. The Cry1Ab protein was below the LOD in pollen (See Table 5) tissue and the PAT protein was below the LOD in kernel and pollen (See Tables 4 and 5) for the plants tested. The eCry3.1Ab and PMI surrogate peptide and labeled surrogate peptide are able to detect eCry3.1Ab and PMI in leaf, kernel, root and pollen from a transgenic corn plant comprising event 5307. The eCry3.1Ab protein was below the LOD in pollen (See Table 5) for the plants tested. The mCry3A and PMI surrogate peptide and labeled surrogate peptide are able to detect mCry3A and PMI proteins in leaf, kernel, root and pollen from a transgenic corn plant comprising event MIR604. The mCry3A protein was below the LOD in pollen (See Table 5) for the plants tested. The Vip3Aa20 and PMI surrogate peptide and labeled surrogate peptide are able to detect Vip3Aa20 and PMI proteins in leaf, kernel, root and pollen from a transgenic corn plant comprising event MIR162. The dmEPSPS surrogate peptide and labeled surrogate peptide are able to detect dmEPSPS protein in leaf, kernel, root and pollen from a transgenic corn plant comprising event GA21.

To further characterize the capability of the preferred labelled surrogate peptides of the invention the assay described above was carried out on a breeding stack expressing all seven proteins, Cry1Ab, eCry3.1Ab, mCry3A, Vip3A, dmEPSPS, PAT and PMI. The results demonstrate that all seven proteins contained in the breeding stack could be detected and quantified concurrently by LC-SRM.

The surrogate peptides and labeled surrogate peptides listed in Table 1 and/or Table 2 are able to detect and/or quantitate target proteins of the invention. Each of these peptides or combination of these peptides are candidates for use in quantitative MRM assays for the target proteins.

TABLE 7
Detection and quantitation of target proteins in transgenic plants.

| Target | | Transgenic Event | | | | |
|---|---|---|---|---|---|---|
| Protein | Tissue | Bt11 | MIR604 | MIR162 | 5307 | GA21 |
| CrylAb | Leaf | 71171 | nd | nd | nd | nd |
| | Kernel | 10480 | nd | nd | nd | nd |
| | Pollen | nd | nd | nd | nd | nd |
| | Root | 87954 | nd | nd | nd | nd |
| eCry3.1Ab | Leaf | nd | nd | nd | 12890 | nd |
| | Kernel | nd | nd | nd | 5771 | nd |
| | Pollen | nd | nd | nd | nd | nd |
| | Root | nd | nd | nd | 5454 | nd |
| mCry3A | Leaf | nd | 12279 | nd | nd | nd |
| | Kernel | nd | 3386 | nd | nd | nd |
| | Pollen | nd | nd | nd | nd | nd |
| | Root | no | 46566 | nd | nd | nd |
| dmEPSPS | Leaf | nd | nd | nd | nd | 6055 |
| | Kernel | nd | nd | nd | nd | 2324 |
| | Pollen | nd | nd | nd | nd | 5745 |
| | Root | nd | nd | nd | nd | 5470 |
| Vip3A | Leaf | nd | nd | 91228 | nd | nd |
| | Kernel | nd | nd | 108110 | nd | nd |
| | Pollen | nd | nd | 29357 | nd | nd |
| | Root | nd | nd | 72530 | nd | nd |
| PAT | Leaf | 17681 | nd | nd | nd | nd |
| | Kernel | nd | nd | nd | nd | nd |
| | Pollen | nd | nd | nd | nd | nd |
| | Root | 17249 | nd | nd | nd | nd |
| PMI | Leaf | nd | 41629 | 105714 | 65349 | nd |
| | Kernel | nd | 88113 | 42800 | 48099 | nd |
| | Pollen | nd | 312531 | 16204 | 312350 | nd |
| | Root | nd | 67112 | 25374 | 22321 | nd |

nd = Not Detected

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth and as follows in scope of the appended claims.

All publications and patent applications mentioned in this specification are indicative of the level of skill of those skilled in the art that this invention pertains. All publications and patent applications are herein incorporated by reference to the same extent as if each individual publication or patent application was specifically and individually indicated to be incorporated by reference.

1. A labeled surrogate peptide that functions in a mass spectrometry assay to selectively detect or quantitate a target transgenic protein selected from the group consisting of a Cry1Ab protein, an eCry3.1Ab protein, a mCry3A protein, a Vip3 protein, a double mutant 5-enolpyruvylshikimate-3-phosphate synthase (dmEPSPS) protein, a phosphinothricin acetyltransferase (PAT) protein and a phosphomannose isomerase (PMI) protein in a mixture of transgenic proteins and non-transgenic proteins in one or more biological samples from one or more transgenic plants, the surrogate peptide comprising a label and an amino acid sequence selected from the group consisting of

| | |
|---|---|
| GSAQGIEGSIR, | (SEQ ID NO: 1) |
| IVAQLGQGVYR, | (SEQ ID NO: 2) |
| TLSSTLYR, | (SEQ ID NO: 3) |
| DVSVFGQR, | (SEQ ID NO: 4) |
| TYPIR, | (SEQ ID NO: 5) |
| TVSQLTR, | (SEQ ID NO: 6) |
| WYNTGLER, | (SEQ ID NO: 7) |
| EWEADPTNPALR, | (SEQ ID NO: 8) |
| VWGPDSR, | (SEQ ID NO: 9) |
| APMFSWIHR, | (SEQ ID NO: 10) |
| WGFDAATINSR, | (SEQ ID NO: 11) |
| NQAISR, | (SEQ ID NO: 12) |
| IEEFAR, | (SEQ ID NO: 13) |
| SGFSNSSVSIIR, | (SEQ ID NO: 14) |
| LSHVSMFR, | (SEQ ID NO: 15) |
| EIYTNPVLENFDGSFR, | (SEQ ID NO: 16) |
| LEGLSNLYQIYAESFR, | (SEQ ID NO: 17) |
| YNQFR, | (SEQ ID NO: 18) |
| YNDLTR, | (SEQ ID NO: 19) |
| SPHLMDILNSITIYTDAHR, | (SEQ ID NO: 20) |
| SAEFNNIIPSSQITQIPLTK, | (SEQ ID NO: 21) |
| QGFSHR, | (SEQ ID NO: 22) |
| MDNNPNINECIPYNCLSNPEVEVLGGER, | (SEQ ID NO: 23) |
| ELTLTVLDIVSLFPNYDSR, | (SEQ ID NO: 24) |
| RPFNIGINNQQLSVLDGTEFAYGTSSNLPSAVYR, | (SEQ ID NO: 25) |
| SGTVDSLDEIPPQNNNVPPR, | (SEQ ID NO: 26) |
| TDVTDYHIDQV, | (SEQ ID NO: 27) |
| AVNELFTSSNQIGLK, | (SEQ ID NO: 28) |
| ITQLPLTK, | (SEQ ID NO: 29) |
| GLDSSTTK, | (SEQ ID NO: 30) |
| QCAGIRPYDGR, | (SEQ ID NO: 31) |
| IEFVPAEVTFEAEYDLER, | (SEQ ID NO: 32) |
| ITQLPLVK, | (SEQ ID NO: 33) |
| MTADNNTEALDSSTTK, | (SEQ ID NO: 34) |
| VYIDK, | (SEQ ID NO: 35) |
| DGGISQFIGDK, | (SEQ ID NO: 36) |
| LITLTCK, | (SEQ ID NO: 37) |
| ELLLATDLSNK, | (SEQ ID NO: 38) |
| FEELTFATETSSK, | (SEQ ID NO: 39) |
| EVLFEK, | (SEQ ID NO: 40) |
| TASELITK, | (SEQ ID NO: 41) |
| DVSEMFTTK, | (SEQ ID NO: 42) |
| LLGLADIDYTSIMNEHLNK, | (SEQ ID NO: 43) |
| IDFTK, | (SEQ ID NO: 44) |
| TDTGGDLTLDEILK, | (SEQ ID NO: 45) |
| DIMNMIFK, | (SEQ ID NO: 46) |
| ALYVHK, | (SEQ ID NO: 47) |
| VNILPTLSNTFSNPNYAK, | (SEQ ID NO: 48) |
| ITSMLSDVIK, | (SEQ ID NO: 49) |
| QNLQLDSFSTYR, | (SEQ ID NO: 50) |
| DSLSEVIYGDMDK, | (SEQ ID NO: 51) |
| MIVEAKPGHALIGFEISNDSITVLK, | (SEQ ID NO: 52) |
| VYFSVSGDANVR, | (SEQ ID NO: 53) |
| NQQLLNDISGK, | (SEQ ID NO: 54) |
| VESSEAEYR, | (SEQ ID NO: 55) |
| YMSGAK, | (SEQ ID NO: 56) |
| DGSPADILDELTELTELAK, | (SEQ ID NO: 57) |
| VYEAK, | (SEQ ID NO: 58) |
| LDAINTMLR, | (SEQ ID NO: 59) |
| GKPSIHLK, | (SEQ ID NO: 60) |
| DENTGYIHYEDTNNNLEDYQTINK, | (SEQ ID NO: 61) |
| DNFYIELSQGNNLYGGPIVHFYDVSIK, | (SEQ ID NO: 62) |
| LLCPDQSEQIYYTNNIVFPNEYVITK, | (SEQ ID NO: 63) |
| SQNGDEAWGDNFIILEISPSEK, | (SEQ ID NO: 64) |
| NAYVDHTGGVNGTK, | (SEQ ID NO: 65) |
| LDGVNGSLNDLIAQGNLNTELSK, | (SEQ ID NO: 66) |
| IANEQNQVLNDVNNK, | (SEQ ID NO: 67) |
| YEVTANFYDSSTGEIDLNK, | (SEQ ID NO: 68) |
| QNYALSLQIEYLSK, | (SEQ ID NO: 69) |
| QLQEISDK, | (SEQ ID NO: 70) |
| LLSPELINTNNWTSTGSTNISGNTLTLYQGGR, | (SEQ ID NO: 71) |
| YVNEK, | (SEQ ID NO: 72) |
| QNYQVDK, | (SEQ ID NO: 73) |
| MAGAEEIVLQPIK, | (SEQ ID NO: 74) |
| FPVEDAK, | (SEQ ID NO: 75) |
| EISGTVK, | (SEQ ID NO: 76) |
| ILLLAALSEGTTVVDNLLNSEDVHYMLGALR, | (SEQ ID NO: 77) |
| DFELPAPPRPVRPVTQI, | (SEQ ID NO: 78) |
| LGLGSTLYTHLLK, | (SEQ ID NO: 79) |
| MSPER, | (SEQ ID NO: 80) |
| HGGWHDVGFWQR, | (SEQ ID NO: 81) |
| NAYDWTVESTVYVSHR, | (SEQ ID NO: 82) |
| TEPQTPQEWIDDLER, | (SEQ ID NO: 83) |
| AAGYK, | (SEQ ID NO: 84) |
| YPWLVAEVEGVVAGIAYAGPWK, | (SEQ ID NO: 85) |
| RPVEIRPATAADMAAVCDIVNHYIETSTVNFR, | (SEQ ID NO: 86) |
| ENAAGIPMDAAER, | (SEQ ID NO: 87) |
| ALAILK, | (SEQ ID NO: 88) |
| SALDSQQGEPWQTIR, | (SEQ ID NO: 89) |
| GSQQLQLKPGESAFIAANESPVTVK, | (SEQ ID NO: 90) |
| FEAKPANQLLTQPVK, | (SEQ ID NO: 91) |
| STLLGEAVAK, | (SEQ ID NO: 92) |
| LINSVQNYAWGSK, | (SEQ ID NO: 93) |
| HNSEIGFAK, | (SEQ ID NO: 94) |
| VLCAAQPLSIQVHPNK, | (SEQ ID NO: 95) |
| TALTELYGMENPSSQPMAELWMGAHPK, | (SEQ ID NO: 96) |
| LSELFASLLNMQGEEK, | (SEQ ID NO: 97) |
| and | |
| QGAELDFPIPVDDFAFSLHDLSDK, | (SEQ ID NO: 98) |
| LQSGASVVAGPR, | (SEQ ID NO: 252) |
| FTTGTDLK | (SEQ ID NO: 255) |
| and | |
| SLTAAVTAAGGNATYVLDGVPR. | (SEQ ID NO: 257) |

2. The labeled surrogate peptide of claim 1, wherein the peptide is labeled by incorporation of a stable isotope labeled (SIL) amino acid.

3. The labeled surrogate peptide of claim 2, wherein the SIL amino acid is lysine, isoleucine, valine or arginine.

4. (canceled)

5. (canceled)

6. (canceled)

7. The surrogate peptide of claim 1, wherein said peptide selectively detects or quantitates an eCry3.1Ab protein and comprises an amino acid sequence selected from the group consisting of TDVTDYHIDQV (SEQ ID NO:27), AVNELFTSSNQIGLK (SEQ ID NO:28), ITQLPLTK (SEQ ID NO:29), GLDSSTTK (SEQ ID NO:30), QCAGIRPYDGR (SEQ ID NO:31) and IEFVPAEVTFEAEYDLER (SEQ ID NO:32).

8. The surrogate peptide of claim 7, wherein said peptide produces a transition ion having an amino acid sequence selected from the group consisting of TDYHIDQV (SEQ ID NO:142), DYHIDQV (SEQ ID NO:143), TSSNQIGLK (SEQ ID NO:144), SSNQIGLK (SEQ ID NO:145), QLPLTK (SEQ ID NO:146), TQLPLTK (SEQ ID NO:147), DSSTTK (SEQ ID NO:148), SSTTK (SEQ ID NO:149), PYDGR (SEQ ID NO:150), DGR, IEF, and LER.

9. The surrogate peptide of claim 7, wherein the peptide comprises the amino acid sequence TDVTDYHIDQV (SEQ ID NO:27) and produces a transition ion consisting of the amino acid sequence TDYHIDQV (SEQ ID NO:142) or DYHIDQV (SEQ ID NO:143).

10. The surrogate peptide of claim 1, wherein said peptide selectively detects or quantitates a mCry3A protein and comprises an amino acid sequence selected from the group consisting of ITQLPLVK (SEQ ID NO:33), MTADNNTEALDSSTTK (SEQ ID NO:34) and VYIDK (SEQ ID NO:35).

11. The surrogate peptide of claim 10, wherein said peptide produces a transition ion having an amino acid sequence selected from the group consisting of QLPLVK (SEQ ID NO:151), TQLPLVK (SEQ ID NO:152), ALDSSTTK (SEQ ID NO:153), EALDSSTTK (SEQ ID NO:154), YIDK (SEQ ID NO:155) and IDK.

12. The surrogate peptide of claim 1, wherein said peptide selectively detects or quantitates a Vip3A protein and comprises an amino acid sequence selected from the group consisting of DGGISQFIGDK (SEQ ID NO:36), LITLTCK (SEQ ID NO:37), ELLLATDLSNK (SEQ ID NO:38), FEELTFATETSSK (SEQ ID NO:39), EVLFEK (SEQ ID NO:40), TASELITK (SEQ ID NO:41), DVSEMFTTK (SEQ ID NO:42), LLGLADIDYTSIMNEHLNK (SEQ ID NO:43), IDFTK (SEQ ID NO:44), TDTGGDLTLDEILK (SEQ ID NO:45), DIMNMIFK (SEQ ID NO:46), ALYVHK (SEQ ID NO:47), VNILPTLSNTFSNPNYAK (SEQ ID NO:48), ITSMLSDVIK (SEQ ID NO:49), QNLQLDSFSTYR (SEQ ID NO:50), DSLSEVIYGDMDK (SEQ ID NO:51), MIVEAKPGHALIGFEISNDSITVLK (SEQ ID NO:52), VYFSVSGDANVR (SEQ ID NO:53), NQQLLNDISGK (SEQ ID NO:54), VESSEAEYR (SEQ ID NO:55), YMSGAK (SEQ ID NO:56), DGSPADILDELTELTELAK (SEQ ID NO:57), VYEAK (SEQ ID NO:58), LDAINTMLR (SEQ ID NO:59), GKPSIHLK (SEQ ID NO:60), DENTGYIHYEDTNNNLEDYQTINK (SEQ ID NO:61), DNFYIELSQGNNLYGGPIVHFYDVSIK (SEQ ID NO:62), LLCPDQSEQIYYTNNIVFPNEYVITK (SEQ ID NO:63), SQNGDEAWGDNFIILEISPSEK (SEQ ID NO:64), NAYVDHTGGVNGTK (SEQ ID NO:65), LDGVNGSLNDLIAQGNLNTELSK (SEQ ID NO:66), IANEQNQVLNDVNNK (SEQ ID NO:67), YEVTANFYDSSTGEIDLNK (SEQ ID NO:68), QNYALSLQIEYLSK (SEQ ID NO:69), QLQEISDK (SEQ ID NO:70), LLSPELINTNNWTSTGSTNISGNTLTLYQGGR (SEQ ID NO:71), YVNEK (SEQ ID NO:72) and QNYQVDK (SEQ ID NO:73).

13. The surrogate peptide of claim 12, wherein said peptide produces a transition ion having an amino acid sequence selected from the group consisting of SQFIGDK (SEQ ID NO:156), GDK, TLTCK (SEQ ID NO:157), TCK, ATDLSNK (SEQ ID NO:158), LATDLSNK (SEQ ID NO:159), TFATETSSK (SEQ ID NO:160), FATETSSK (SEQ ID NO:161), FEK, LFEK (SEQ ID NO:162), SELITK (SEQ ID NO:163), ASELITK (SEQ ID NO:164), SEMFTTK (SEQ ID NO:165), DVS, IMNEHLNK (SEQ ID NO:166), MNEHLNK (SEQ ID NO:167), DFTK (SEQ ID NO:168), FTK, TLDEILK (SEQ ID NO:169), LTLDEILK (SEQ ID NO:170), MNMIFK (SEQ ID NO:171), NMIFK (SEQ ID NO:172), YVHK (SEQ ID NO:173), HK, VNI, VNIL (SEQ ID NO:174), SMLSDVIK (SEQ ID NO:175), TSMLSDVIK (SEQ ID NO:176), DSFSTYR (SEQ ID NO:177), LDSFSTYR (SEQ ID NO:178), IYGDMDK (SEQ ID NO:179), VIYGDMDK (SEQ ID NO:180), SNDSITVLK (SEQ ID NO:181), MIV, SGDANVR (SEQ ID NO:182), SVSGDANVR (SEQ ID NO:183), LLNDISGK (SEQ ID NO:184), LNDISGK (SEQ ID NO:185), SSEAEYR (SEQ ID NO:186), ESSEAEYR (SEQ ID NO:187), SGAK (SEQ ID NO:188), MSGAK (SEQ ID NO:189), TELTELAK (SEQ ID NO:190), DGSPADI (SEQ ID NO:191), YEAK (SEQ ID NO:192), EAK, NTMLR (SEQ ID NO:193), AINTMLR (SEQ ID NO:194), PSIHLK (SEQ ID NO:195), HLK, DYQTINK (SEQ ID NO:196), NK, DNF, DNFY (SEQ ID NO:197), PNEYVITK (SEQ ID NO:198), LLC, SPSEK (SEQ ID NO:199), LEISPSEK (SEQ ID NO:200), NAY, DHTGGVNGTK (SEQ ID NO:201), GNLNTELSK (SEQ ID NO:202), NTELSK (SEQ ID NO:203), LNDVNNK (SEQ ID NO:204), NDVNNK (SEQ ID NO:205), YE, DLNK (SEQ ID NO:206), QIEYLSK (SEQ ID NO:207), LQIEYLSK (SEQ ID NO:208), SDK, QEISDK (SEQ ID NO:209), YQGGR (SEQ ID NO:210), TLYQGGR (SEQ ID NO:211), NEK, VNEK (SEQ ID NO:212), DK, and VDK.

14-35. (canceled)

36. The surrogate peptide of claim 1, wherein the mixture of transgenic proteins comprises at least two transgenic proteins selected from the group consisting of a Cry1Ab protein, a eCry3.1Ab protein, a mCry3A protein, a Vip3A protein, a dmEPSPS protein, a PAT protein and a PMI protein.

37. (canceled)

38. (canceled)

39. The surrogate peptide of claim 1, wherein the transgenic plant is selected from the group consisting of corn, soybean, cotton, rice, wheat, canola and eggplant.

40. (canceled)

41. (canceled)

42. (canceled)

43. The surrogate peptide of claim 1, wherein the biological sample is from leaf tissue, seed, grain, pollen, or root tissue.

44. (canceled)

45. (canceled)

46. An assay cassette comprising at least two labeled surrogate peptides of claim 1.

47. A method of simultaneously detecting or quantitating one or more target transgenic proteins in a complex biological sample from a transgenic plant comprising a mixture of the target transgenic proteins and non-transgenic proteins, the method comprising:
a. obtaining a biological sample from a transgenic plant;
b. extracting proteins from the biological sample, resulting in an extract comprising a mixture of proteins;
c. reducing the amount of non-transgenic insoluble proteins in the extract of step b, resulting in an extract of concentrated soluble proteins;
d. digesting the soluble proteins in the extract of step c, resulting in an extract comprising peptide fragments, wherein the peptide fragments include at least one surrogate peptide specific for each target transgenic protein;
e. concentrating the peptide fragments in the extract of step d,
f. adding one or more labeled surrogate peptides of claim 1, wherein each labeled surrogate peptide has the same amino acid sequence as each surrogate peptide of the target transgenic proteins, and wherein the number of labeled surrogate peptides that are added is equal to the number of target transgenic proteins in the mixture;
g. concentrating the surrogate peptides and the labeled surrogate peptides by reducing the amount of non-surrogate peptides in the mixture;
h. resolving the peptide fragment mixture from step g via liquid chromatography;
i. analyzing the peptide fragment mixture resulting from step h via mass spectrometry, wherein detection of a transition ion fragment of a labeled surrogate peptide is indicative of the presence of a target transgenic protein from which the surrogate peptide is derived; and optionally,
j. calculating an amount of a target transgenic protein in the biological sample by comparing mass spectrometry signals generated from the transition ion fragment of step i with mass spectrometry signals generated by a transition ion of a labeled surrogate peptide.

48. The method of claim 47, wherein the target transgenic protein is a Cry1Ab protein, a eCry3.1Ab protein, a mCry3A protein, a Vip3 protein, a double mutant 5-enolpyruvylshikimate-3-phosphate synthase (dmEPSPS) protein, a phosphinothricin acetyltransferase (PAT) protein or a phosphomannose isomerase (PMI) protein.

49. (canceled)

50. (canceled)

51. The method of claim 47, wherein the target transgenic protein is an eCry3.1Ab protein and the labeled surrogate peptide comprises the amino acid sequence TDVTDYHIDQV (SEQ ID NO:27) and produces a transition ion consisting of the amino acid sequence TDYHIDQV (SEQ ID NO:142) or DYHIDQV (SEQ ID NO:143).

52. The method of claim 51, wherein the eCry3.1Ab transgenic protein is quantitated in the biological sample by comparing mass spectrometry signals generated from a transition ion fragment consisting of the amino acid sequence

53. (canceled)

54. (canceled)

55. (canceled)

56. The assay cassette of claim 46, wherein the at least two labeled surrogate peptides comprise at least two of SAEFNNIIPSSQITQIPLTK (SEQ ID NO:21), TDVTDYHIDQV (SEQ ID NO: 27), LQSGASVVAGPR (SEQ ID 252), FTTGTDLK (SEQ ID NO: 255), SLTAAVTAAGGNATYVLDGVPR (SEQ ID NO: 257), LGLGSTLYHLLK (SEQ ID NO: 79) or SALDSQQGEPWQTIR (SEQ ID NO:89).

57. The assay cassette of claim 56, wherein the at least two labeled surrogate peptides comprise SAEFNNIIPSSQITQIPLTK (SEQ ID NO:21), TDVTDYHIDQV (SEQ ID NO: 27), LQSGASVVAGPR (SEQ ID 252), FTTGTDLK (SEQ ID NO: 255), SLTAAVTAAGGNATYVLDGVPR (SEQ ID NO: 257), LGLGSTLYHLLK (SEQ ID NO: 79) and SALDSQQGEPWQTIR (SEQ ID NO:89)
